SEQUENCE LISTING (5 sequences)

SEQ ID NO: 1 - protein, 10 aa, Artificial sequence
  Myc-tag
EQKLISEEDL

SEQ ID NO: 2 - DNA, 29 bp, Artificial sequence
  PCR primer
cggggtaccatgatcatcttaatttactt

SEQ ID NO: 3 - DNA, 24 bp, Artificial sequence
  PCR primer
ggtgatcagtggctaaatcttcca

SEQ ID NO: 4 - DNA, 29 bp, Artificial sequence
  PCR primer
ggagtactctttgcgtgtgggttgtagca

SEQ ID NO: 5 - protein, 277 aa, Homo sapiens
  TSG-6 protein
MIILIYLFLLLWEDTQGWGFKDGIFHNSIWLERAAGVYHREARSGKYKLTYAEAKAVCEFEGGHLATYKELEAARKIGFHVCAAGWMAKGRVGYPIVKPGPNCGFGKTGIIDYGIRLNRSERWDAYCYNPHAKECGGVFTDPKEIFKSPGFPNEYEDNQICYWHIRLKYGQRIHLSFLDFDLEDDPGCLADYVEIYDSYDDVHGFVGRYCGDELPDDIISTGNVMTLKFLSDASVTAGGFQIKYVAMDPVSKSSQGKNTSTTSTGNKNFLAGRFSHL

(19) United States
(12) Patent Application Publication
(10) Pub. No.: US 2016/0075750 A1
(43) Pub. Date: Mar. 17, 2016

(54) Production of TSG-6 Protein

(71) Applicants: Darwin J. Prockop, Philadelphia, PA (US); Jun Watanabe, Kanagawa (JP); Dong-Ki Kim, Temple, TX (US); Rwang Hwa Lee, Temple, TX (US); Hosoon Choi, Belton, TX (US)

(72) Inventors: Darwin J. Prockop, Philadelphia, PA (US); Jun Watanabe, Kanagawa (JP); Dong-Ki Kim, Temple, TX (US); Rwang Hwa Lee, Temple, TX (US); Hosoon Choi, Belton, TX (US)

(21) Appl. No.: 14/793,086

(22) Filed: Jul. 7, 2015

Related U.S. Application Data
(63) Continuation-in-part of application No. 14/354,381, filed on Apr. 25, 2014.
(60) Provisional application No. 61/555,681, filed on Nov. 4, 2011.

(51) Int. Cl. C07K 14/525

(52) U.S. Cl. CPC C07K 14/525

(57) ABSTRACT

A method of producing a protein or polypeptide, such as, for example, TSG-6 protein, or a biologically active fragment, derivative or analogue thereof, by introducing into mammalian cells a polynucleotide encoding the biologically active protein or polypeptide or biologically active fragment, derivative, or analogue thereof. The cells then are suspended in a protein-free medium that includes at least one agent that suppresses production of hyaluronic acid, hyaluronan, or a salt thereof by the cells. The cells are cultured for a time sufficient to express the biologically active protein or polypeptide or biologically active fragment, derivative or analogue thereof. The biologically active protein or polypeptide, or fragment, derivative, or analogue thereof then is recovered from the cells, such as, for example, by recovering the protein or polypeptide secreted by the cells from the cell culture medium.

This application is a continuation-in-part of application Ser. No. 14/354,381 filed Apr. 25, 2014 which is the national phase application of PCT Application No. PCT/US12/62985 Filed Nov. 1, 2012, which claims priority based on provisional application Ser. No. 61/555,681, filed Nov. 4, 2011, the contents of which are incorporated by reference in their entireties.

This invention relates to the production of proteins or polypeptides such as, for example, TSG-6 protein, by mammalian cells. More particularly, this invention relates to the production of such proteins, and biologically active fragments, derivatives, and analogues thereof by introducing into mammalian cells a polynucleotide encoding a biologically active protein or polypeptide, or a biologically active fragment, derivative, or analogue thereof, and then culturing the cells by suspending the cells in a protein-free medium that includes at least one agent suppresses the production of hyaluronic acid or hyaluronan or a salt thereof by the cells. The cells are cultured for a period of time sufficient to express the biologically active protein or polypeptide, or a biologically active fragment, derivative, or analogue thereof. The biologically active protein or polypeptide, or a biologically active fragment, derivative, or analogue thereof then is recovered from the cultured cells.

Biologically active proteins and polypeptides, as well as fragments, derivatives, or analogues thereof, have a variety of therapeutic uses. Examples of such biologically active proteins and polypeptides include, but are not limited to, anti-inflammatory proteins, such as, for example, tumor necrosis factor stimulated gene 6 protein, or TSG-6, protein, anti-apoptotic proteins, such as, for example, stanniocalcin-1 and stanniocalcin-2, or STC-1 and STC-2, proteins, proteins that regulate cell growth and development, such as, for example, LIF protein; proteins that regulate hematopoiesis, such as, for example, IL-11, proteins that kill cancer cells or regulate immune response, such as, for example, TNFSF10 (also known as TRAIL), and IL-24; proteins that regulate homing of cells, such as, for example, CXCR4; proteins involved in cell adhesion and cell signaling, such as, for example, ITGA2 (also known as integrin α2); and proteins that enhance angiogenesis, such as, for example, IL-8.

Such biologically active proteins have a variety of therapeutic uses. For example, the anti-inflammatory protein, TSG-6, may be used to treat diseases and disorders of the eye, including dry eye syndrome (See U.S. Pat. No. 9,062,103), macular degeneration, including age related macular degeneration (ARMD), and other maculopathies and retinal degeneration, corneal injury (See U.S. Pat. No. 8,785,395), corneal diseases and disorders, diseases and disorders of the anterior chamber of the eye, diseases and disorders of the iris; lens, and retina, eyelid diseases, lacrimal apparatus diseases, and glaucoma. TSG-6 also may be used to treat inflammation associated with myocardial infarction, stroke, Alzheimer's disease, atherosclerosis, and lung diseases.

Furthermore, TSG-6 may be used to treat inflammation associated with autoimmune diseases and immune pathologies, including rheumatoid arthritis, bacterial and/or viral infection, chronic inflammatory pathologies, vascular inflammatory pathologies, neurodegenerative disease, malignant pathologies involving TNF-secreting tumors, and alcohol-induced hepatitis. (See, for example, U.S. Pat. Nos. 6,210,905 and 6,313,091).

TSG-6 protein is a multifunctional endogenous protein that is expressed by a variety of cells in response to stimulation by pro-inflammatory cytokines (Fulop, et al, Gene, Vol. 202, pgs. 95-102 (1997); Milner, et al., Biochem. Soc. Transactions, Vol. 34, pgs. 446-450 (2006); Szanto, et al., Arthritis and Rheumatism, Vol. 50, pgs. 3012-3022 (2004); Wisniewski, et al., Cytokine and Growth Factor Reviews, Vol. 15, pg. 129-146 (2004)). The protein has a molecular weight of about 35 kDa and consists primarily of an N-terminal link domain similar to the hyaluronan-binding module of proteoglycans, and a C-terminal domain with sequences similar to complement C1r/C1s, an embryonic sea urchin growth factor Uegf and BMP1 (CUB domain) (Blundell, et al., J. Biol. Chem., Vol. 280, pgs. 18189-18201 (2005)). TSG-6 binds to a large number of components of the extracellular matrix including hyaluronan, heparin, heparan sulfate, thrombospondins-1 and -2, fibronectin, and pentraxin (Blundell, 2005; Baranova, et al., J. Biol. Chem., Vol. 286, pgs. 25675-25686 (2011); Kuznetsova, et al., Matrix Biology, Vol. 27, pgs. 201-210 (2008); Mahoney et al., J. Biol. Chem., Vol. 280, pgs. 27044-27055 (2005)). These interactions primarily act to stabilize the extracellular matrix.

In addition, TSG-6 modulates inflammatory responses by several effects, some of which are related to its stabilization of extracellular matrix but some of which appear to be independent. One of the more complex interactions is that the protein catalytically transfers the heavy chains of inter-α-trypsin inhibitor onto hyaluronan (Rugg et al., J. Biol. Chem., Vol. 280, pgs. 25674-25686 (2005)). It thereby helps stabilize the extracellular matrix. Simultaneously, it releases the bikunin component from inter-α-trypsin inhibitor to increase its activity in inhibiting the cascade of proteases released during inflammatory responses (Okroj et al., J. Biol. Chem., Vol. 287, pgs. 20100-20110 (2012); Scavenius, et al., Biochim. et Biophys. Acta, Vol. 1814, pgs. 1624-1630 (2011); Zhang, et al., J. Biol. Chem., Vol. 287, pgs. 12433-12444 (2012)). In apparently independent actions, TSG-6 reduces the migration of neutrophils through endothelial cells (Cao, et al., Microcirculation, Vol. 11, pgs. 615-624 (2004)), forms a ternary complex with murine mast cell trypases and heparin (Nagyeri, et al., J. Biol. Chem., Vol. 286, pgs. 23559-23569 (2011)), and inhibits FGF-2 induced angiogenesis through an interaction with pentraxin (Leali, et al., Arteriosclerosis, Thrombosis, and Vascular Biology, Vol. 32, pgs. 696-703 (2012)). In addition, TSG-6 either directly or through a complex with hyaluronan, binds to CD44 on resident macrophages in a manner that decreases TLR2/NF-κB signaling and modulates the initial phase of the inflammatory response of most tissues (Oh, et al., Molecular Therapy, Vol. 20, pgs. 2143-2152 (2012); Oh, et al., Proc. Nat. Acad. Sci., Vol. 107, pgs. 16875-16880 (2010)’ Choi, et al., Blood, Vol. 118, pgs. 330-338 (2011)). TSG-6 thereby reduces the large, second phase of inflammation that frequently is an excessive and deleterious response to sterile injuries (Prockop, et al., Molecular Therapy, Vol. 20, pgs. 14-20 (2012)).

These and related observations stimulated interest in the therapeutic potentials of the TSG-6. For example, transgenic mice with localized over-expression of the gene in joints or cartilage had a decreased response to experimentally-induced arthritis (Glant, et al., Arthritis and Rheumatism, Vol. 46, pgs. 2207-2218 (2002); Mindrescu, et al., Arthritis and Rheumatism, Vol. 46, pgs. 2453-2464 (2002)). Conversely, mice with a knock-out of the gene had increased susceptibility to proteoglycan-induced arthritis (Szanto, 2004). Also, administration of recombinant TSG-6 decreased experimentally-induced arthritis in several different models (Bardos, et al., Am. J. Pathology, Vol. 159, pgs. 1711-1721 (2001); Mindrescu, et al., Arthritis and Rheumatism, Vol. 43, pgs. 2668-2677 (2000)). In addition, the recombinant protein decreased osteoblastogenesis and osteoclast activity (Mahoney et al., J. Biol. Chem., Vol. 283, pgs. 25952-25962 (2008); Mahoney, et al, Arthritis and Rheumatism, Vol. 63, pgs. 1034-1043 (2011)). Interest in the therapeutic potentials of the protein was increased further by the recent observations that enhanced expression of the protein by adult stem/progenitor cells referred to as mesenchymal stem/stromal cells (MSCs) explained some of the beneficial effects observed after administration of the cells in animal models for myocardial infarction (Lee et al., Cell Stem Cell, Vol. 5, pgs. 54-63 (2009)), chemical injury to the cornea (Oh, 2012; Oh, 2010), zymosan-induced peritonitis (Choi, 2011), and LPS-induced or bleomycin-induced lung injury (Szanto, 2004; Danchuk, et al., Stem Cell Research and Therapy, Vol. 2, pg. 27 (2011)).

The therapeutic proteins hereinabove described may be produced by a variety of techniques known to those skilled in the art, such as, for example, recombinant or genetic engineering techniques. For example, appropriate cells, such as, for example, mammalian cells or insect cells, may be genetically engineered with a polynucleotide that encodes a biologically active protein or polypeptide, or a biologically active fragment, derivative, or analogue thereof. The cells then are cultured under conditions such that the cells express the biologically active protein or polypeptide, or a biologically active fragment, derivative, or analogue thereof.

Although biologically active proteins and polypeptides may be produced by recombinant techniques, some biologically active proteins and polypeptides, such as TSG-6, for example, are produced in limited quantities, and/or are difficult to recover from the cells which produce such proteins. Indeed, the ability to produce TSG-6 protein in sufficient amounts, to surmount the technical complexities, and to do so in a cost effective manner and efficiently has limited further study and development of TSG-6 protein, and of therapies employing TSG-6 protein.

### BRIEF DESCRIPTION OF THE DRAWINGS

The invention now will be described with respect to the drawings.

FIGS. 1A and 1B. Transiently transfected Chinese Hamster Ovary (CHO) cells express hTSG-6 wild-type and hTSG-6-LINK proteins. (FIG. 1A) Diagram of expression constructs encoding hTSG-6 wild-type protein or hTSG-6-LINK protein with a His-tag that are inserted into a pEF4/Myc-His expression vector. Each cDNA was fused to six histidine codons at its C-terminus under the control of a human elongation factor promoter (P_{EF}-1α). (FIG. 1B(i) and (ii)) After two days post-transfection, the transformed cells were labeled with anti-TSG and anti-His antibodies.

FIGS. 2A through 2D. Rapid establishment of hTSG-6/CHO stable cell lines using a methylcellulose-based formulation. (FIG. 2A) The cells were evaluated under a microscope at 0, 3, 7, and 14 days post-transfection. At 14 days post-transfection, the transformed clones that formed spheres were isolated under a microscope. (FIG. 2B) About 50 clones were analyzed for hTSG-6 protein secretion by an ELISA assay as a first screening step. Absorbance was measured at 450 nm. (FIG. 2C) Selected clones were analyzed for hTSG-6 protein secretion by a Western Blot assay. (FIG. 2D) The most productive clones were amplified further and as a final test, the expression of TSG-6 proteins within the clones was verified by immunocytochemistry with a fluorescent-labeled hTSG-6 antibody.

FIGS. 3A through 3F. Determination of optimal medium for spinner culture of rhTSG-6/CHO stable cell lines in chemical-defined protein free media supplemented with various factors. (FIGS. 3A-E) (FIG. 3F) The optimal medium (Sup A) provided greater viability and survival than the standard CD-CHO medium.

FIGS. 4A and 4B. Cell growth and TSG-6 yields in a bioreactor using the optimal medium (FIG. 3F). Cell seeding density was 5×10⁴ cells/ml. (FIG. 4A) 34° C. (FIG. 4B) 36° C.

FIGS. 5A through 5C. Large scale purification of rhTSG-6 and its link module, hTSG-6-LINK- (FIG. 5A) protein purification steps of the cultured media of stable CHO cell lines. (FIGS. 5B and 5C) SDS-PAGE profile of protein fractions. Multiple bands are detected with hTSG-6-LINK because of varying degrees of glycosylation.

FIGS. 6A through 6C. rhTSG-6 and rhTSG-6-LINK reduced corneal opacity and inflammation in the cornea following, injury. Corneas of rats were injured by 15 second exposure to ethanol followed by mechanical scraping of the epithelium and limbus, (Oh, et al., Proc. Nat. Acad. Sci., Vol. 107, No. 39, pgs. 16875-16880 (2010)). (FIG. 6A). Corneal opacity was reduced significantly in both rhTSG-6 and rhTSG-6-LINK-treated corneas. (FIG. 6B) For a quantitative measure of neutrophil infiltration, the concentration of myeloperoxidase (MPO) was assayed. Treatment with either rhTSG-6 or rhTSG-6-LINK reduced the levels of MPO in the cornea significantly. (FIG. 6C) The protein levels of the proinflammatory cytokine IL-1β were decreased significantly in the corneas treated with rhTSG-6 or rhTSG-6 LINK as assayed by ELISA.

FIGS. 7A through 7F. CHO cell line stably transduced to synthesize rhTSG-6 forms aggregates resulting in a decrease of protein production. Cells were incubated in CD CHO basal media and in spinner culture. (FIG. 7A. i) Diagram of expression plasmid of TSG-6 under control of human elongation factor promoter 1α and insertion of sequences for Myc- and His-tags at the C-terminus. (FIG. 7A. ii) Expression of rhTSG-6 and the His-tag by immunocytochemistry. (FIG. 7A. iii) Expression by Western blotting. (FIG. 7B.) Number of control CHO cells (CHO-S) and rhTSG-6 synthesizing cells (rhTSG-6/CHO-S). (FIG. 7C.) rhTSG-6 content and pH of medium from cultures of rhTSG-6/CHO-S cells. (FIG. 7D). Western blots with antibodies to the His tag of medium from cultures of rhTSG-6/CHO-S cells. (FIG. 7E.) Aggregates of rhTSG-6/CHO-S cells after 4 days of culture. Scale bar=500 μm. (FIG. 7F.) Immunolabeling with rhTSG-6 antibody (clone A38.1.20), HABP (biotin-conjugated HA binding proteins), and DAPI of aggregates. Scale bar=100 μm.

FIGS. 8A through 8D. Rapid generation of stable clones of rhTSG-6/CHO using a methylcellulose-based protocol. (FIG. 8A.) Schematic diagram for the generation of the clones. (FIG. 8B.) Phase contrast photographs of transfected clones of CHO cells. The cloned CHO cells formed spheres that were up to 500 μm in diameter. (FIG. 8C.) Western blots with antibodies to rhTSG-6 (arrows) in medium from stable clones. (FIG. 8D.) Immunocytochemistry of an isolated clone labeled with antibodies to rhTSG-6.

FIGS. 9A through 9D. Addition of heparin to medium improved yield of rhTSG-6 in spinner cultures. (FIG. 9A.) Effect on number of rhTSG-6/CHO-S cells. (FIG. 9B.) Effect on yield of rhTSG-6. (FIG. 9C.) Effect on yields of protein aggregates/complexes (H-rhTSG-6) and monomeric rhTSG-6 (L-rhTSG-6). Western blots with antibodies to hTSG-6 on medium from 4 day cultures of rhTSG-6/CHO-S cells. (FIG. 9D.) Effects on pH of medium.

FIGS. 10A through 10D. Optimal conditions for culture of stably transfected CHO cells in a chemically defined and protein-free medium. The cells were cultured in 500 mL of medium in spinner bottle cultures. (FIG. 10A.) Effects of increasing glucose concentration to 11 mM. All subsequent trials were with 11 mM glucose. (FIG. 10B.) Effects of adding non-essential amino acids (cat#11140-050; Invitrogen). (FIG. 10C.) Effect of adding a lipid concentrate (cat#11905-031; Invitrogen) and a surfactant (Pluronic F-68; Invitrogen) either separately or together. (FIG. 10D.) Effect of culture with the optimized chemically-defined and protein-free medium (OCDPF medium) that was developed on the basis of the trial experiments.

FIGS. 11A through 11D. Synthesis of rhTSG-6 by culture of rhTSG-6/CHO-S cells in OCDPF medium and in a bioreactor that controlled pH. (FIG. 11A.) Expansion of cells and oxygen saturation. (FIG. 11B.) Yield of rhTSG-6 and pH of medium. (FIG. 11C.) Stability of rhTSG-6/CHO-S cells. Scale bar=100 μm (FIG. 11D.) Yield of monomeric rhTSG-6. Western blot of medium with antibodies to hTSG-6.

FIGS. 12A through 12D. Purification of rhTSG-6 from 5 L cultured media of the bioreactor. (FIG. 12A.) Schematic for the purification steps. (FIG. 12B.) Assay by gel electrophoresis of rhTSG-6 eluted from the Q-sepharose column. Gel was stained with Coomassie Blue. (FIG. 12C.) Endotoxin content of conditioned culture medium, eluate from the His-tag column and Q-sepharose column. (FIG. 12D.) Deglycosylation of the purified rhTSG-6 (black arrows indicate glycosylated rhTSG-6 and gray arrow indicates deglycosylated rhTSG-6).

FIG. 13. In vivo half-life of rhTSG-6 proteins in plasma of mice. rhTSG-6 proteins (50 μg) were injected through the tail vein and the blood was collected at times indicated. After separation of the plasma, levels of rhTSG-6 proteins were determined by ELISA. Distribution (t_{1/2α}) and elimination (t_{1/2β}) were calculated using GraphPad Prism program. Myeloma-derived rhTSG-6: t_{t/2α}=0.15 hr, t_{1/2β}=0.20 hr, CHO cell-derived rhTSG-6: t_{1/2α}, =0.08 hr, t_{1/2β}=0.47 hr.

FIGS. 14A through 14C. Purified rhTSG-6 suppressed LPS-induced inflammation in mice. (FIG. 14A.) Schematic for the experiment. (FIG. 14B.) rhTSG-6 suppressed LPS-induced levels of mRNA for IL-6 in spleen. (FIG. 14C.) rhTSG-6 suppressed LPS-induced levels of mRNA for IFNγ in spleen.

### DETAILED DESCRIPTION OF THE INVENTION

It therefore is an object of the present invention to provide a more efficient method of producing recombinant biologically active proteins and polypeptides, and to produce such biologically active proteins and polypeptides in greater quantities.

In accordance with an aspect of the present invention, there is provided a method of producing a biologically active protein or polypeptide, or a biologically active fragment, derivative, or analogue thereof. The method comprises introducing into cells, including, but not limited to, mammalian cells, a polynucleotide encoding a biologically active protein or polypeptide, or a biologically active fragment, derivative, or analogue thereof. The cells then are cultured by suspending the cells in a protein-free medium that includes at least one agent that suppresses production of hyaluronic acid or hyaluronan or a salt thereof by the cells. The cells are cultured for a time sufficient to express the biologically active protein or polypeptide, or a biologically active fragment, derivative, or analogue thereof. The expressed biologically active protein or polypeptide, or a biologically active fragment, derivative, or analogue thereof then is recovered from the cells.

Applicants have discovered that, if the medium also includes an agent that inhibits or prevents the aggregation of the genetically engineered cells, that such cells express greater amounts of the biologically active protein or polypeptide, or biologically active fragment, derivative, or analogue thereof. Thus, in a non-limiting embodiment, the medium further includes at least one agent that inhibits or prevents the aggregation of the cells. Agents that inhibit or prevent the aggregation of the cells include, but are not limited to, heparin, dextran sulfate, ferric citrate, and combinations thereof. In another non-limiting embodiment, the agent that inhibits or prevents the aggregation of the cells is heparin.

In an alternative non-limiting embodiment, the medium in which the cells are cultured may contain protein, provided that the protein which is present does not interfere with the growth of the cultured cells, or interfere with optimal production of the biologically active protein or polypeptide, or biologically active fragment, derivative, or analogue thereof.

Thus, in accordance with another aspect of the present invention, there is provided a method of producing a biologically active protein or polypeptide, or a biologically active fragment, derivative, or analogue thereof. The method comprises introducing into cells, such as mammalian cells, a polynucleotide encoding a biologically active protein or polypeptide, or a biologically active fragment, derivative, or analogue thereof. The cells then are cultured by suspending the cells in a medium that includes at least one agent that suppresses production of hyaluronic acid or hyaluronan or a salt thereof by the cells. The cells are cultured for a time sufficient to express the biologically active protein or polypeptide, or a biologically active fragment, derivative, or analogue thereof. The expressed biologically active protein or polypeptide, or a biologically active fragment, derivative, or analogue thereof then is recovered from the cells.

In a non-limiting embodiment, the medium further includes at least one agent that inhibits or prevents the aggregation of the cells, such as, for example, heparin, dextran sulfate, ferric citrate, and combinations thereof, as hereinabove described.

In a non-limiting embodiment, the biologically active protein or polypeptide, or a biologically active fragment, derivative, or analogue thereof is a biologically active protein or polypeptide having a link domain or link module.

In another non-limiting embodiment, the biologically active protein or polypeptide is TSG-6 protein, or biologically active fragment, derivative, or analogue thereof. In another non-limiting embodiment, the biologically active protein or polypeptide includes the TSG-6 protein hyaluronan-binding link domain. The sequence of the “native” TSG-6 protein, having 277 amino acid residues, is given in the example hereinbelow. In one non-limiting embodiment, the link domain consists of amino acid residues 1 through 133. In another non-limiting embodiment, the link domain consists of amino acid residues 1 through 98, as described in Day, et al. Protein Expr. Purif., Vol. 1, pgs. 1-16 (Aug. 8, 1996).

The inflammation-associated protein TSG-6 cross-links hyaluronan via hyaluronan-induced TSG-6 oligomers. (Baranova, (2011). Tumor necrosis factor-stimulated gene 6 (TSG-6) is a hyaluronan-binding protein that plays important roles in inflammation and ovulation. TSG-6-mediated cross-linking of hyaluronan (HA) has been proposed as a functional mechanism (e.g., for regulating leukocyte adhesion) but direct evidence for cross-linking has been lacking. Full-length TSG-6 protein binds with pronounced positive cooperativity and it can cross-link HA at physiologically relevant concentrations. Cooperative binding of full-length TSG-6 arises from HA-induced protein oligomerization, and the TSG-6 oligomers act as cross-linkers. In contrast, the HA-binding domain of TSG-6 (i.e., the link module) alone binds without positive cooperativity and binds more weakly than the full-length protein. Both the link module and full-length TSG-6 protein condensed and rigidified HA films, and the degree of condensation scaled with the affinity between the TSG-6 constructs and HA. The condensation may be the result of protein-mediated HA cross-linking. TSG-6 is a potent HA cross-linking agent and may have important implications for the mechanistic understanding of the biological functions of TSG-6.

In another non-limiting embodiment, the biologically active protein or polypeptide or a biologically active fragment, derivative, or analogue thereof, such as TSG-6 protein or biologically active fragment, derivative, or analogue thereof, has a “His-tag” at the C-terminal thereof. The term “His-tag”, as used herein, means one or more histidine residues are bound to the C-terminal of the TSG-6 protein or biologically active fragment, derivative, or analogue thereof. In another non-limiting embodiment, the “His-tag” has six histidine residues at the C-terminal of the biologically active protein or polypeptide, such as TSG-6 protein or a biologically active fragment, derivative, or analogue thereof.

In a non-limiting embodiment, when the biologically active protein or polypeptide, or biologically active fragment, derivative, or analogue thereof, includes a “His-tag”, at the C-terminal thereof, the biologically active protein or polypeptide, or biologically active fragment, derivative, or analogue thereof, may include a cleavage site that provides for cleavage of the “His-tag” from the biologically active protein or polypeptide, or biologically active fragment, derivative, or analogue thereof, after the biologically active polypeptide, or biologically active fragment, derivative, or analogue thereof is produced.

In another non-limiting embodiment, the biologically active protein or polypeptide, or a biologically active fragment, derivative, or analogue thereof, such as TSG-6 protein or a biologically active fragment, derivative, or analogue thereof, has a “Myc-tag” at the N-terminal or C-terminal thereof. The term “Myc-tag”, as used herein, means a polypeptide tag derived from the c-myc gene product. In a non-limiting embodiment, the “Myc-tag” has the amino acid sequence EQKLISEEDL.

In a non-limiting embodiment, when the biologically active protein or polypeptide, or biologically active fragment, derivative, or analogue, thereof, includes a “Myc-tag” at the N-terminal or C-terminal thereof, the biologically active protein or polypeptide, or biologically active fragment, derivative, or analogue thereof, may include a cleavage site that provides for cleavage of the “Myc-tag” from the biologically active protein or polypeptide, or biologically active fragment, derivative, or analogue thereof, after the biologically active polypeptide, or biologically active fragment, derivative, or analogue thereof is produced.

The polynucleotide that encodes the biologically active polypeptide, or a biologically active fragment, derivative, or analogue thereof may be a DNA or RNA. Such polynucleotides include all nucleotides that are degenerate versions of each other and that encode the same amino acid sequence. The polynucleotide may include introns.

In general, the polynucleotide encoding the biologically active protein or polypeptide, or a biologically active fragment, derivative, or analogue thereof is part of a gene construct in which the polynucleotide encoding, the biologically active protein or polypeptide, or a biologically active fragment, derivative, or analogue thereof is linked operatively to regulatory sequences to achieve expression of the polynucleotide in the mammalian cell. Such regulatory sequences including typically a promoter and a polyadenylation signal.

In a non-limiting embodiment, the gene construct is provided as an expression vector that includes the coding sequence for the biologically active protein or polypeptide which is linked operably to essential regulatory sequences such that when the vector is transfected into the cell, the coding sequence will be expressed by the mammalian cell. The coding sequence is linked operably to the regulatory elements necessary for expression of that sequence in the mammalian cells. The nucleotide sequence that encodes the biologically active protein or polypeptide may be cDNA, genomic DNA, synthesized DNA or a hybrid thereof, or an RNA molecule such as mRNA.

The gene construct includes the nucleotide sequence encoding the biologically active protein or polypeptide, which is linked operably to the regulatory elements and may remain present in the mammalian cell as a functioning cytoplasmic molecule, a functioning episomal molecule, or it may integrate into the mammalian cell's chromosomal DNA. Exogenous genetic material may be introduced into the cells where it remains as separate genetic material in the form of a plasmid. Alternatively, linear DNA which can integrate into the chromosome may be introduced into the mammalian cell. When introducing DNA into the mammalian cell, reagents which promote DNA integration into chromosomes may be added. DNA sequences which are useful to promote integration may also be included in the DNA molecule. Alternatively, RNA may be introduced into the mammalian cell.

The regulatory elements for gene expression include: a promoter, an initiation codon, a stop codon, and a polyadenylation signal. It is preferred that these elements be operable in the mammalian cells of the present invention. Moreover, it is preferred that these elements be linked operably to the nucleotide sequence that encodes the protein or polypeptide such that the nucleotide sequence can be expressed in the cells and thus the protein can be produced. Initiation codons and stop codons are considered generally to be part of a nucleotide sequence that encodes the protein or polypeptide; however, it is preferred that these elements are functional in the mammalian cells. Similarly, promoters and polyadenylation signals used must be functional within the cells of the present invention. Examples of promoters useful to practice the present invention include, but are not limited to, promoters that are active in many cells such as the cytomegalovirus promoter, SV40 promoters, and retroviral promoters. In some non-limiting embodiments, promoters are used which express genes in the mammalian cells constitutively with or without enhancer sequences. Enhancer sequences are provided in such embodiments when appropriate or desirable.

In a non-limiting embodiment, the polynucleotide encoding the biologically active protein or polypeptide, or biologically active fragment, derivative, or analogue thereof is contained in a pEF4/Myc-His expression vector. (Invitrogen). Such vectors include a human elongation factor 1a-subunit (hEF-1α) promoter which controls expression of the polynucleotide encoding the biologically active protein or polypeptide or a biologically active fragment, derivative, or analogue thereof, a multiple cloning site, a C-terminal tag encoding a polyhistidne (6 histidne residues) metal binding polypeptide, a Zeocin resistance gene flanked by an SV40 origin of replication and an SV40 poly A signal, and an ampicillin resistance gene.

The mammalian cells of the present invention can be transfected using well known techniques readily available to those having ordinary skill in the art. Exogenous genes may be introduced into the cells using standard methods where the cell expresses the protein encoded by the gene. In some embodiments, mammalian cells are transfected by calcium phosphate precipitation transfection, DEAE dextran transfection, electroporation, microinjection, liposome-mediated transfer, chemical-mediated transfer, ligand mediated transfer or recombinant viral vector transfer.

In some non-limiting embodiments, recombinant adenovirus vectors are used to introduce DNA with desired sequences into the mammalian cell. In some non-limiting embodiments, recombinant retrovirus vectors are used to introduce DNA with desired sequences into the mammalian cells. In other embodiments, standard CaPO₄, DEAE dextran or lipid carrier mediated transfection techniques are employed to incorporate desired DNA into dividing mammalian cells. In some non-limiting embodiments, DNA is introduced directly into the mammalian cells by microinjection. Similarly, well-known electroporation or particle bombardment techniques can be used to introduce foreign DNA into the cells. A second gene may be co-transfected with, or linked to the polynucleotide encoding the biologically active protein or polypeptide. The second gene frequently is a selectable marker, such as a selectable antibiotic-resistance gene. Standard antibiotic resistance selection techniques can be used to identify and select transfected biologically active protein or polypeptide cells. Transfected cells are selected by growing the cells in an antibiotic that will kill cells that do not take up the selectable gene. In most cases where the two genes co-transfected and unlinked, the cells that survive the antibiotic treatment contain and express both genes.

In another non-limiting embodiment, the polynucleotide encoding the biologically active protein or polypeptide is contained in an expression cassette, and is linked operably to a suitable promoter.

The expression cassette containing the polynucleotide encoding the biologically active protein or polypeptide should be incorporated into the genetic vector suitable for delivering the transgene to the mammalian cell. Depending on the desired end application, any such vector can be so employed to modify the cells genetically (e.g., plasmids, naked DNA, viruses such as adenovirus, adeno-associated virus, herpesvirus, lentivirus, papillomavirus, retroviruses, etc.). Any method of constructing the desired expression cassette within such vectors can be employed, many of which are well known in the art, such as by direct cloning, homologous recombination, etc. The desired vector will determine largely the method used to introduce the vector into the cells, which are generally known in the art. Suitable techniques include protoplast fusion, calcium-phosphate precipitation, gene gun, electroporation, and infection with viral vectors.

Mammalian cells which may be employed include any mammalian cell into which may be introduced a polynucleotide encoding a biologically active protein or polypeptide, or a biologically active fragment, derivative, or analogue thereof. In a non-limiting embodiment, the mammalian cells are Chinese hamster ovary, or CHO, cells.

Alternatively, the polynucleotide encoding a biologically active protein or polypeptide, or biologically active fragment, derivative, or analogue thereof, may be introduced into other eukaryotic ells, such as yeast cells, or prokaryotic cells, such as E. coli cells, for example.

The cells which include the polynucleotide encoding the biologically active protein or polypeptide are suspended in an appropriate protein-free medium that includes at least one agent that suppresses production of hyaluronic acid or hyaluronan or a salt thereof by the cells.

In a non-limiting embodiment, the at least one agent that suppresses production of hyaluronic acid or hyaluronan or a salt thereof by the mammalian cells is 4-methylumbelliferone, also known as MU or 7-hydroxy-4 methyl-2H-1-benzopyran-2-one. Although the scope of the present invention is not to be limited to any theoretical reasoning, certain biologically active proteins or polypeptides, such as TSG-6 and fragments, derivatives, or analogues thereof, bind to hyaluronic acid or hyaluronan or a salt thereof, produced by the cells, and thus are secreted by the cell in reduced quantities. By suppressing the production of hyaluronic acid or hyaluronan or a salt thereof, the 4-methylumbelliferone may enable the cells to produce and secrete increased amounts of the biologically active protein or polypeptide, such as TSG-6 protein or a biologically active fragment, derivative, or analogue thereof, or may allow higher synthesis, or better recovery and separation of the biologically active protein or polypeptide from the cells.

In other non-limiting embodiments, the at least one agent that suppresses production of hyaluronic acid or hyoluronan or a salt thereof by the cells is an antisense polynucleotide or small interfering RNA (siRNA) that blocks hyaluronan synthesis, or an antibody that binds to hyaluronan.

In another non-limiting embodiment, the protein-free medium is free of plasma.

In a further non-limiting embodiment, the protein-free medium includes chemically defined CHO medium, hypoxanthine/thymine, or HT, L-glutamine, glucose (such as, for example, D-(+)-glucose), 4-methylumbelliferone, non-essential amino acids, MEM (Minimal Essential Medium) vitamin solution, penicillin, and streptomycin.

The cells are cultured under conditions and for a time sufficient to express the biologically active protein or a biologically active fragment, derivative, or analogue thereof in a desired amount. In a non-limiting embodiment, the cells are cultured at a temperature of about 36° C. In another non-limiting embodiment, the cells are cultured for a total period of time of from about 2 days to about 14 days. In yet another non-limiting embodiment, the cells are cultured for a total period of time of from about 4 days to about 10 days.

In a non-limiting embodiment, the cells are transfected with a pEF4/Myc-His vector which includes the polynucleotide encoding a biologically active protein or polypeptide or fragment, derivative, or analogue thereof. The transfected cells then are plated onto a medium containing fetal bovine serum (MS) and Iscove's Modified Dulbecco's Medium, (IMDM), and Zeocin. The cells are cultured until they reach a cell density of about 90%.

The cells then are cultured in a spinner bottle, whereby the cells are suspended in a protein-free medium such as hereinabove described, and which includes at least one agent, e.g., 4-methylumbelliferone, that suppresses production of hyaluronic acid by the cells. The cells are cultured at a temperature of 36° C. until they reach an appropriate cell density, such as, for example, about 0.3 to 60×10⁴ cells/ml. In a non-limiting embodiment, such period of time is about 4 days.

The cells then are suspended in the protein-free medium, such as hereinabove described, in a bioreactor. A pH control reagent, such as NaOH, may be added to the medium to maintain the pH of the medium at about 7.4. The cells are cultured in the bioreactor until they reach an appropriate cell density, such as, for example, about 175-220×10⁴ cells/ml. In a non-limiting embodiment, such period of time is at least about 5 days.

The cultured medium then is collected from the bioreactor, and the biologically active protein or polypeptide, such as TSG-6 protein or a biologically active fragment, derivative, or analogue thereof, such as a TSG-6 protein having a His-tag of 6 histidine residues at the C-terminal thereof, is recovered from the cultured medium. Such recovery may be effected by any of a variety of means known to those skilled in the art. Such methods include, but are not limited to, ion exchange gradient columns used in combination with an appropriate buffer, and the like. When the protein or polypeptide includes a His-tag at the C-terminal thereof, a column containing a nickel chelate His-tag resin also may be employed as part of the protein recovery process.

The biologically active proteins or polypeptides, or a biologically active fragments, derivatives, or analogues thereof, that are produced and recovered in accordance with the present invention, may be employed in their respective therapeutic uses. For example, in a non-limiting embodiment, TSG-6 protein, or TSG-6 protein or biologically active fragment, derivative, or analogue thereof, including TSG-6 protein or fragment, derivative, or analogue thereof that includes a “His-tag” at the C-terminal thereof, may be used in any of the therapeutic applications hereinabove described for TSG-6 protein, including the treatment of diseases or disorders of the eye.

Applicants have discovered that, when TSG-6 protein, or a biologically active fragment, derivative, or analogue thereof, includes a “His-tag” at the C-terminal thereof, such TSG-6 protein or a fragment, derivative, or analogue thereof having a “His-tag” at the C-terminal thereof, has the same biological activity as a “native” TSG-6 protein or biologically active fragment, derivative, or analogue thereof.

For example, the TSG-6 protein or biologically active fragment, derivative, or analogue thereof, including TSG-6 protein having a His-tag at the C-terminal thereof, may be used to treat various ocular diseases or conditions, including the following: maculopathies/retinal degeneration: macular degeneration, including age related macular degeneration (ARMD), such as non-exudative age related macular degeneration and exudative age related macular degeneration, choroidal neovascularization, retinopathy, including diabetic retinopathy, acute and chronic macular neuroretinopathy, central serous chorioretinopathy, and macular edema, including cystoid macular edema, and diabetic macular edema. Uveitis/retinitis/choroiditis: acute multifocal placoid pigment epitheliopathy, Behcet's disease, birdshot retinochoroidopathy, infectious (syphilis, Lyme Disease, tuberculosis, toxoplasmosis), uveitis, including intermediate uveitis (pars planitis) and anterior uveitis, multifocal choroiditis, multiple evanescent white dot syndrome (MEWDS), ocular sarcoidosis, posterior scleritis, serpignous choroiditis, subretinal fibrosis, uveitis syndrome, and Vogt-Koyanagi-Harada syndrome. Vascular diseases/exudative diseases: retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, central retinal artery occlusion, branch retinal artery occlusion, carotid artery disease (CAD), frosted branch angitis, sickle cell retinopathy and other hemoglobinopathies, angioid streaks, familial exudative vitreoretinopathy, Eales disease, Traumatic/surgical: sympathetic ophthalmia, uveitic retinal disease, retinal detachment, trauma, laser, PDT, photocoagulation, hypoperfusion during surgery, radiation retinopathy, bone marrow transplant retinopathy. Proliferative disorders: proliferative vitreal retinopathy and epiretinal membranes, proliferative diabetic retinopathy. Infectious disorders: ocular histoplasmosis, ocular toxocariasis, presumed ocular histoplasmosis syndrome (PONS), endophthalmitis, toxoplasmosis, retinal diseases associated with HIV infection, choroidal disease associated with HIV infection, uveitic disease associated with HIV Infection, viral retinitis, acute retinal necrosis, progressive outer retinal necrosis, fungal retinal diseases, ocular syphilis, ocular tuberculosis, diffuse unilateral subacute neuroretinitis, and myiasis. Genetic disorders: retinitis pigmentosa, systemic disorders with associated retinal dystrophies, congenital stationary night blindness, cone dystrophies, Stargardt's disease and fundus flavimaculatus, Best's disease, pattern dystrophy of the retinal pigmented epithelium, X-linked retinoschisis, Sorsby's fundus dystrophy, benign concentric maculopathy, Bietti's crystalline dystrophy, pseudoxanthoma elasticum. Retinal tears/holes: retinal detachment, macular hole, giant retinal tear. Tumors: retinal disease associated with tumors, congenital hypertrophy of the RPE, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, intraocular lymphoid tumors. Miscellaneous: punctate inner choroidopathy, acute posterior multifocal placoid pigment epitheliopathy, myopic retinal degeneration, acute retinal pigment epithelitis and the like.

An anterior ocular condition is a disease, ailment or condition which affects or which involves an anterior (i.e. front of the eye) ocular region or site, such as a periocular muscle, an eyelid or an eyeball tissue or fluid which is located anterior to the posterior wall of the lens capsule or ciliary muscles. Thus, an anterior ocular condition primarily affects or involves the conjunctiva, the cornea, the anterior chamber, the iris, the posterior chamber (behind the retina but in front of the posterior wall of the lens capsule), the lens or the lens capsule and blood vessels and nerve which vascularize or innervate an anterior ocular region or site.

Thus, an anterior ocular condition can include a disease, ailment or condition, such as for example, aphakia; pseudophakia; astigmatism; blepharospasm; cataract; conjunctival diseases; conjunctivitis, including, but not limited to, atopic keratoconjunctivitis; corneal injuries, including, but not limited to, injury to the corneal stromal areas; corneal diseases; corneal ulcer; dry eye syndromes; eyelid diseases; lacrimal apparatus diseases; lacrimal duct obstruction; myopia; presbyopia; pupil disorders; refractive disorders and strabismus. Glaucoma can also be considered to be an anterior ocular condition because a clinical goal of glaucoma treatment can be to reduce a hypertension of aqueous fluid in the anterior chamber of the eye (i.e. reduce intraocular pressure).

A posterior ocular condition is a disease, ailment or condition which primarily affects or involves a posterior ocular region or site such as choroid or sclera (in a position posterior to a plane through the posterior wall of the lens capsule), vitreous, vitreous chamber, retina, optic nerve (i.e. the optic disc), and blood vessels and nerves which vascularize or innervate a posterior ocular region or site. Thus, a posterior ocular condition can include a disease, ailment or condition, such as for example, acute macular neuroretinopathy; Behcet's disease; choroidal neovascularization; diabetic uveitis; histoplasmosis; infections, such as fungal or viral-caused infections; macular degeneration, such as acute macular degeneration, non-exudative age related macular degeneration and exudative age related macular degeneration; edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic opthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, non-retinopathy diabetic, retinal dysfunction, retinitis pigmentosa, and glaucoma. Glaucoma can be considered a posterior ocular condition because the therapeutic goal is to prevent the loss of or reduce the occurrence of loss of vision due to damage to or loss of retinal cells or optic nerve cells (i.e. neuroprotection).

Other diseases or disorders of the eye which may be treated with the TSG-6 protein or biologically active fragment, derivative, or analogue thereof, including a TSG-6 protein or biologically active fragment, derivative, or analogue thereof having a His-tag of 6 amino acid residues at the C-terminal thereof, include, but are not limited to, ocular cicatricial pemphigoid (OCP), and cataracts.

In a non-limiting embodiment, when inflammation of and/or injury to and/or disease or disorder of the eye is associated with an infection, e.g., a bacterial, viral, or fungal infection, the TSG-6 protein or biologically active fragment, derivative, or analogue thereof may be administered in combination with at least one anti-infective agent.

In general, at least one anti-infective agent which is administered in combination with the TSG-6 protein or biologically active fragment, derivative, or analogue thereof depends upon the type of infection, e.g., bacterial, viral, or fungal, to the eye, the type or species of bacterium, virus, or fungus associated with the infection, and the extent and severity of the infection, and the age, weight, and sex of the patient.

In a non-limiting embodiment, when the infection of the eye is associated with one or more bacteria, the at least one anti-infective agent which is administered in combination with the TSG-6 protein or biologically active fragment, derivative, or analogue thereof is at least one anti-bacterial agent. Anti-bacterial agents which may be administered include, but are not limited to, quinolone antibiotics, such as, for example, ciprofloxacin, levofloxacin (Cravit), moxifloxacin (Vigamox), gatifloxacin (Zy-mar), cephalosporin, aminoglycoside antibiotics (e.g., gentamycin), and combinations thereof.

In another non-limiting embodiment, when the infection of the eye is associated with one or more viruses, the anti-infective agent which is administered in combination with the TSG-6 protein or biologically active fragment, derivative, or analogue thereof is at least one anti-viral agent. Anti-viral agents which may be employed include those which are known to those skilled in the art.

In another non-limiting embodiment, when the infection of the eye is associated with one or more fungi, the anti-infective agent which is administered in combination with the TSG-6 protein or biologically active fragment, derivative, or analogue thereof is at least one anti-fungal agent. Anti-fungal agents which may be employed include, but are not limited to, natamycin, amphotericin B, and azoles, including fluconazole and itraconzole.

In yet another non-limiting embodiment, when the infection of the eye is associated with more than one of bacteria, viruses, and fungi, more than one of anti-bacterial, anti-viral, and anti-fungal agents are administered in combination with the TSG-6 protein or biologically active fragment, derivative, or analogue thereof.

In a non-limiting embodiment, the TSG-6 protein or biologically active fragment, derivative, or analogue thereof may be administered to a patient in combination with other therapeutic agents employed in treating macular degeneration. Such therapeutic agents include, but are not limited to, angiogenesis inhibitors, and anti-vascular endothelial growth factor A (VEGF-A) antibodies (eg., Avastin, Lucentis), agents or drugs which bind angiogenic agents, such as VEGF trap agents, tyrosine kinase inhibitors, which are anti-angiogenic, angiogenic protein receptor antagonists, and antibodies and antibody fragments which recognize heat shock proteins, including, but not limited to antibodies and antibody fragments which recognize the small heat shock protein HSPB4, HSP90, HSP70, HSP65, or HSP27, and heat shock protein antagonists, including, but not limited to, antagonists to HSPB4, HSP90, HSP70, HSP65, and HSP27.

Administration of the TSG-6 protein or biologically active fragment or derivative or analogue thereof typically is parenteral, by intravenous, subcutaneous, intramuscular, or intraperitoneal injection, or by infusion or by any other acceptable systemic method. In a non-limiting embodiment, the TSG-6 protein or biologically active fragment, derivative, or analogue thereof is provided to a mammal by intraocular administration. In a non-limiting embodiment, administration is by intravenous infusion, typically over a time course of about 1 to 5 hours. In addition, there are a variety of oral delivery methods for the administration of the TSG-6 protein or biologically active fragment, derivate or analogue thereof.

Alternatively, in a non-limiting embodiment, the TSG-6 protein or biologically active fragment, derivative, or analogue thereof may be administered to the eye topically, such as, for example, in the form of eye drops. In a further non-limiting embodiment, eye drops which include the TSG-6 protein or an analogue or fragment or derivative thereof, are administered to the cornea in order to treat or prevent a disease or disorder of the cornea.

In another non-limiting embodiment, the TSG-6 protein or biologically active fragment, derivative, or analogue thereof may be administered systemically, such as by intravenous administration, or intraocularly, such as by intracameral administration, i.e., to the anterior chamber of the eye.

Often, treatment dosages are titrated upward from a low level to optimize safety and efficacy. Generally, daily dosages will fall within a range of about 0.01 to 20 mg protein per kilogram of body weight. Typically, the dosage range will be from about 0.1 to 5 mg protein per kilogram of body weight.

Various modifications or derivatives of the TSG-6 protein or biologically active fragment, derivative, or analogue thereof, such as addition of polyethylene glycol chains (PEGylation), may be made to influence their pharmacokinetic and/or pharmacodynamic properties.

To administer the TSG-6 protein or biologically active fragment, derivative, or analogue thereof, by other than parenteral administration, the protein may be coated or co-administered with a material to prevent its inactivation. For example, the TSG-6 protein or biologically active fragment, derivative or analogue thereof, may be administered in an incomplete adjuvant, co-administered with enzyme inhibitors or administered in liposomes. Enzyme inhibitors include pancreatic trypsin inhibitor, disopropylfluorophosphate (DEP) and trasylol. Liposomes include water-in-oil-in-water, CGF emulsions, as well as conventional liposomes (Strejan, et al., (1984) J. Neuroimmunol. 7:27).

An “effective amount” of the TSG-6 protein or biologically active fragment, derivative, or analogue thereof, is an amount that will ameliorate one or more of the well known parameters that characterize medical conditions such as inflammation associated with the cornea, as well as the other diseases and disorders of the eye hereinabove described. An effective amount, in the context of inflammatory diseases of the cornea, as well as the other diseases or disorders hereinabove described, is the amount of protein or fragment, derivative, or analogue thereof that is sufficient to accomplish one or more of the following: decrease the severity of symptoms; decrease the duration of disease exacerbations; increase the frequency and duration of disease remission/symptom-free periods; prevent fixed impairment and disability; and/or prevent/attenuate chronic progression of the disease.

Although the compositions of this invention can be administered in simple solution, they are more typically used in combination with other materials such as carriers, preferably pharmaceutical carriers. Useful pharmaceutical carriers can be any compatible, non-toxic substance suitable for delivering the compositions of the invention to a patient. Sterile water, alcohol, fats, waxes, and inert solids may be included in a carrier. Pharmaceutically acceptable adjuvants (buffering agents, dispersing agents) may also be incorporated into the pharmaceutical composition. Generally, compositions useful for parenteral administration of such drugs are well known; e.g., Remington's Pharmaceutical Science, 17th Ed. (Mack Publishing Company, Easton, Pa., 1990). Alternatively, compositions of the invention may be introduced into a patient's body by implantable drug delivery systems [Urquhart et al., Ann. Rev. Pharmacol. Toxicol. 24:199 (1984).

Therapeutic formulations may be administered in many conventional dosage formulations. Formulations typically comprise at least one active ingredient, together with one or more pharmaceutically acceptable carriers.

The formulations conveniently may be presented in unit dosage form and may be prepared by any methods well known in the art of pharmacy. See, e.g., Gilman et al. (eds.) (1990), The Pharmacological Bases of Therapeutics, 8th Ed., Pergamon Press; and Remington's Pharmaceutical Sciences, supra, Easton, Pa.; Avis, et al. (eds.) (1993) Pharmaceutical Dosage Forms: Parenteral Medications Dekker, N.Y.; Lieberman et al. (eds.) (1990), Pharmaceutical Dosage Forms: Tablets, Dekker, N.Y.; and Lieberman et al. (eds.) (1990), Phamiaceutical Dosage Forms: Disperse Systems, Dekker, N.Y.

Therapeutic compositions and formulations thereof of the invention can be used, for example, for reducing inflammation due to seasonal or bacterial conjunctivitis, for reducing post-surgical pain and inflammation, to prevent or treat fungal or bacterial infections of the eye, to treat herpes ophthalmicus, to reduce intraocular pressure, or to treat endophthalmitis.

More particularly, in one non-limiting embodiment, the present invention provides a method for treating an ophthalmic disorder in a mammal (e.g., including human and non-human primates), the method comprising administering to the eye of the mammal a therapeutically effect amount of a formulation of the present invention comprising a lipid phase, an aqueous phase and a TSG-6 protein or biologically active fragment, derivative, or analogue thereof as hereinabove described, wherein the protein or biologically active fragment, derivative, or analogue thereof, is useful for treating the ophthalmic disorder. In one embodiment, the ophthalmic disorder is post-operative pain. In another embodiment, the ophthalmic disorder is ocular inflammation resulting from, e.g., iritis, conjunctivitis, seasonal allergic conjunctivitis, acute and chronic endophthalmitis, anterior uveitis, uveitis associated with systemic diseases, posterior segment uveitis, chorioretinitis, pars planitis, masquerade syndromes including ocular lymphoma, pemphigoid, scleritis, keratitis, severe ocular allergy, corneal abrasion and blood-aqueous barrier disruption. In yet another embodiment, the ophthalmic disorder is post-operative ocular inflammation resulting from, for example, photorefractive keratectomy, cataract removal surgery, intraocular lens implantation and radial keratotomy.

In employing the liposome formulations of the present invention, in a non-limiting embodiment, administration is ocularly, which term is used to mean delivery of therapeutic agents through the surface of the eye, including the sclera, the cornea, the conjunctiva and the limbus, or into the anterior chamber of the eye. Ocular delivery can be accomplished by numerous means, for example, by topical application of a formulation such as an eye drop, by injection, or by means of an electrotransport drug delivery system.

In another non-limiting embodiment, the TSG-6 protein or biologically active fragment, derivative, or analogue thereof employed for treating a disease or disorder of the eye may be contained in a nanoparticle. Such nanoparticles may be formed by methods known to those skilled in the art.

Such nanoparticles may be administered ocularly, i.e., through the surface of the eye, including the sclera, cornea, conjunctiva, and the limbus, or into the anterior chamber of the eye. Such ocular administration may be accomplished by any of a variety of means, including, in a non-limiting embodiment, by topical application of a formulation such as an eye drop, by injection, or by means of an electotransport drug delivery system.

### EXAMPLES

The invention now will be described with respect to the following examples; it is to be understood, however, that the scope of the present invention is not intended to be limited thereby.

### Example 1

### Material and Methods

hMSCs Culture

Frozen vials of human mesenchymal stem cells (hMSCs) from bone marrow were obtained from the Center for the Preparation and Distribution of Adult Stem Cells (formerly http://www.com.tulane.edu/genetherapy/distribute.shtml; currently http://medicine.tamhsc.edu/irm/msc-distribution.html) that supplies standardized preparations of MSCs enriched for early progenitor cells to over 300 laboratories under the auspices of an NIH/NCRR grant (P40 RR 17447-06). A frozen vial of 10⁶ passage 1 cells was thawed, and plated at 200 to 500 cells/cm² in 150 mm plates with 30 mL of complete culture medium (CCM) that consisted of α-minimal essential medium (α-MEM; Invitrogen, Carlsbad, Calif.), 17% fetal bovine serum (FBS; lot-selected for rapid growth of MSCs; Atlanta Biologicals, Inc., Norcross, Ga.), 100 units/mL penicillin, 100 μg/mL streptomycin, and 2 mM L-glutamine (Invitrogen). The cultures were incubated for approximately 5 days until they were 70% confluent with replacement of medium every 2 days. The cultures were washed with PBS and the cells harvested by incubation for 5 to 10 min. at 37° C. with 0.25% trypsin and 1 mM EDTA.

In order to up-regulate expression of TSG-6, the MSCs were expanded to about 70% confluency and then incubated at 37° C. for 24 hours in α-MEM containing 20 ng/mL TNF-α, 2% FBS, 100 units/mL penicillin, 100 μg/mL streptomycin, and 2 mM L-glutamine (Lee et al., Cell Stem Cell, Vol. 5, pgs. 54-63 (2009)).

### Plasmid Construction

Total RNA was isolated from TNF-α stimulated hMSC cells (3×10⁴ cells/cm²) and one microgram of total RNA was used to produce the first strand cDNA pool by RT-PCR (Superscript II/oligo dT₁₂₋₁₈, Invitrogen). cDNA encoding hTSG-6 (GenBank accession number: NM_{—}007115) was amplified by PCR. Primer sequences for the hTSG-6 genes that were cloned were 5′-CGGGGTACCATGATCATCTTAATTTACTT-3′ (sense for hTSG-6-WT and -LINK), 5′-GGTGATCAGTGGCTAAATCTTCCA-3′ (anti-sense for hTSG-6-WT), and 5′-GGAGTACTCTTTGCGTGTGGGTTGTAGCA-3′ (antisense for hTSG-6-LINK). The TSG-6 protein has the following amino acid sequence shown below. The TSG-6-LINK protein, or TSG-6 link module domain, consists of amino acid residues 1 through 133 hereinbelow:

MIILIYLFLL LWEDTQGWGF KDGIFHNSIW LERAAGVYHR
EARSGKYKLT YAEAKAVCEF EGGHLATYKQ LEAARKIGFH
VCAAGWMAKG RVGYPIVKPG PNCGFGKTGI IDYGIRLNRS
ERWDAYCYNP HAKECGGVFT DPKQIFKSPG FPNEYEDNQI
CYWHIRLKYG QRIHLSFLDF DLEDDPGCLA DYVEIYDSYD
DVHGFVGRYC GDELPDDIIS TGNVMTLKFL SDASVTAGGF
QIKYVAMDPV SKSSQGKNTS TTSTGNKNFL AGRFSHL

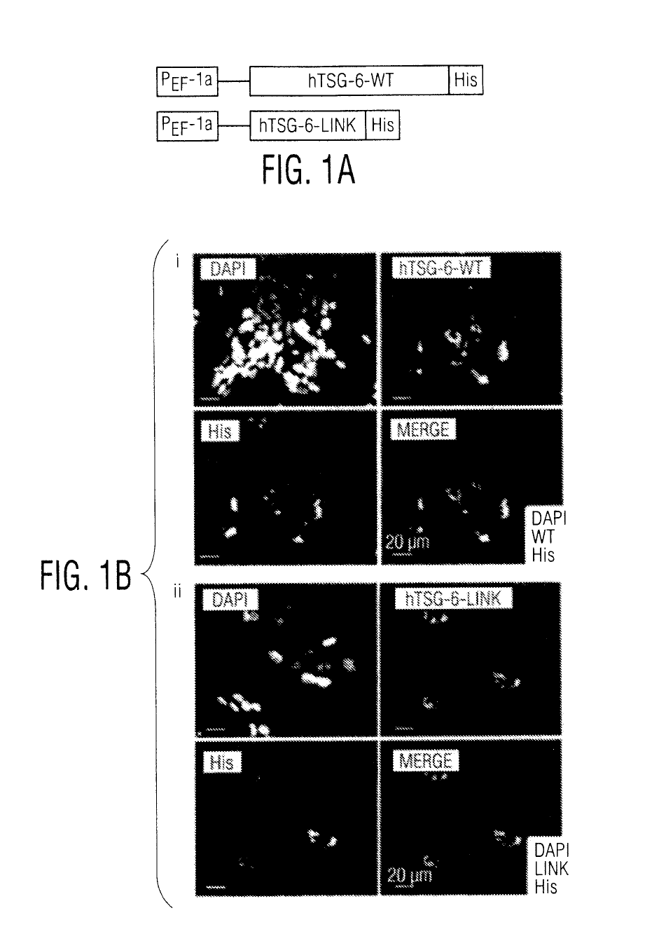

The PCR products were subcloned into the BamHI and EcoRI sites in the multiple cloning site of a pEF4/Myc/His plasmid (Invitrogen, Carlsbad, Calif.). Thus, the resulting pEF4/Myc-His plasmid vectors include DNA encoding hTSG-6 wild-type(WT) or hTSG-6-LINK protein under the control of the P_{EF}-1α promoter, each of which has a DNA sequence encoding a His-tag of 6 histidine residues at the 3′ end. (FIG. 1A).

### Establishment of rh TSG-6-WT and -LINK/CHO Stable Cell Lines

Chinese Hamster Ovary (CHO)-S cells were plated at 1×10⁵ cells in a 100 mm culture dish in 10 mL IMDM (Iscove's Modified Dulbecco's Medium) containing 5% FBS, 50 units/ml of penicillin, and 50 μg/ml of streptomycin. After incubation for 2 days, cells were transfected with 30 μg of the constructed expression vector for rhTSG-6-WT or rhTSG-6-LINK using 20 μl of Lipofectamine 2000™ (invitrogen) in serum-reduced Opti media (Invitrogen). Four hours later, the medium was replaced with 10 ml of 5% FBS/IMDM and further incubated for one day. In order to determine whether the cells were expressing TSG-6 or TSG-6-LINK protein, the cells were labeled with DAPI and fluorescent antibodies which bind to TSG-6 or histidine. As shown in FIGS. 1B(i) and (ii), it was determined that the transfected cells expressed TSG-6 or TSG-6-LINK protein. The next day, the transfected cells were lifted and reseeded in a 100 mm culture dish in 9 mL ClonaCell-TCS medium (StemCell technologies) containing 500 μg/ml of Zeocin to select transformed clones. The cells were cultured further for 14 days, a time sufficient for the clones to form spheres in the methylcellulose-based semi-solid selection media.

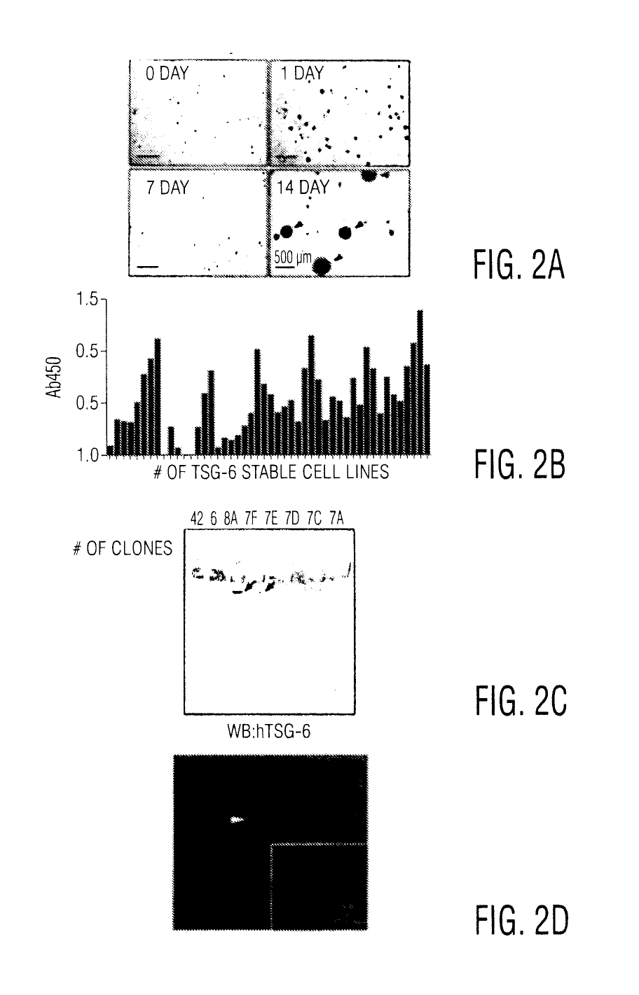

The clones were examined under a microscope at 0, 3, 7, and 14 days post-transfection. After 14 days post-transfection, the transformed clones that form spheres were isolated under a microscope using a pipette. (FIG. 2A). About 50 clones then were tested and analyzed for TSG-6 protein secretion by ELISA, in which absorbance was measured at 450 nm. (FIG. 2B). Selected clones, i.e., clones 42, 6, 8A, 7F, 7E, 7D, 7C, and 7A, then were analyzed for TSG-6 protein secretion by Western Blot. (FIG. 2C). The most productive clones then were amplified further by plating on 15 cm diameter dishes in CCM and culturing for 2 days, and as a final test, the expression of TSG-6 protein within the clones was verified by immunocytochemistry with a fluorescent-labeled anti hTSG-6 antibody. (FIG. 2D).

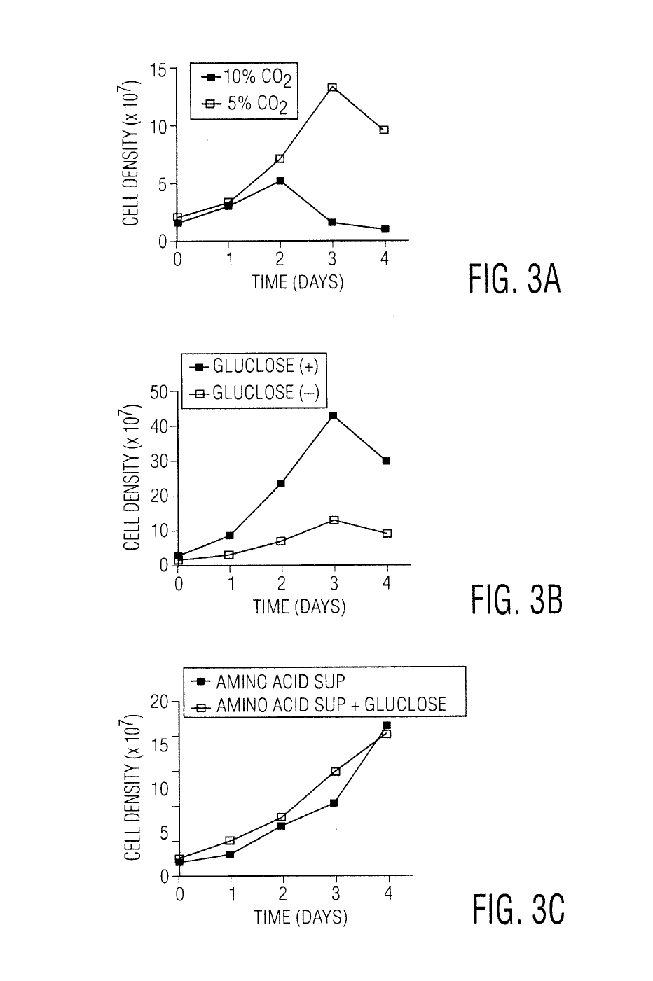

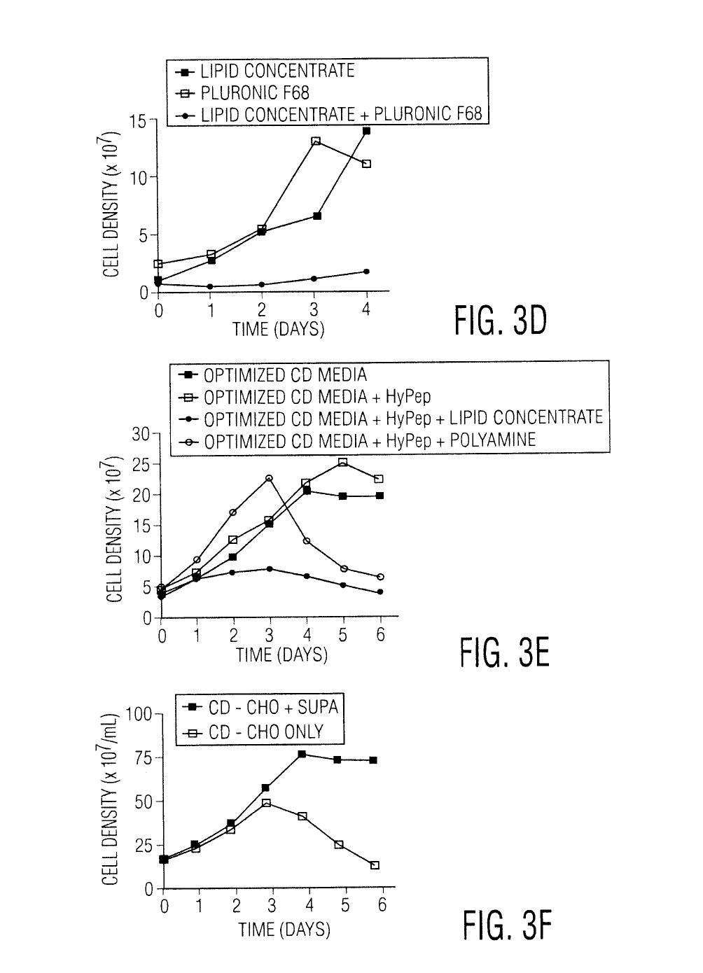

The optimal medium for culturing rhTSG-6/CHO cell lines was determined by incubating the cell lines in a spinner bottle by seeding the cells in a chemically defined protein free medium (CDPF) that included 1 liter of CHO medium (CD-CHO, cat. #10743-011; Invitrogen), either alone (FIG. 3F), or in combination with 5% or 10% CO₂ (FIG. 3A); D-(+)-glucose or D-(−)-glucose (FIG. 3B); 10 ml non-essential amino acids or non-essential amino acids in combination with glucose (FIG. 3C); lipid concentrate, Pluronic F68, or lipid concentrate and Pluronic F68 (FIG. 3D); 10 ml hypoxanthine/thymidine medium (HT 100×, or HyPep cat. #11067-030, Invitrogen), or Hy Pep and lipid concentrate, or Hy Pep and polyamine (FIG. 3E). As indicated in FIG. 3F, the cells also were cultured in a medium referred to as CD-CHO+SupA, which is a chemically defined protein free medium (CDPF) that was prepared with 1 liter CHO medium (CD-CHO cat. #10743-011; Invitrogen), 10 mL hypoxathine/thymidine medium (HT 100×, cat. #11067-030; Invitrogen), 40 mL L-glutamine (final concentration 8 mM; L-Glutamine 200 mM; cat. #G6152-100G; Sigma); 2 grams D-(+)-glucose (cat. # G6152-100G; Sigma). 10 mL non-essential amino acids (cat. #11140-050; Invitrogen), 10 mL MEM vitamin solution (cat. #11120-052; Invitrogen), 5 mL penicillin/streptomycin (10,000 units Penicillin and 10,000 μg Streptomycin; cat. #15140163; Invitrogen) and 4-methylumbelliferone added to a 50 μM concentration (Wako Pure Chemicals; Osaka, Japan).

The cells were cultured in the various media hereinabove described for a period of time of from 4 days to 6 days, after which cell densities were measured. As shown in FIG. 3F, the cells that were cultured in the CD−CHO+Sup A medium had greater viability and survival than cells cultured in the other media shown in FIGS. 3A through 3E.

The most productive clones were expanded in a spinner bottle by seeding about 3×10⁴ cells/mL in 500 mL in 5 liters of CDPF medium (i.e., CD−CHO+Sup A).

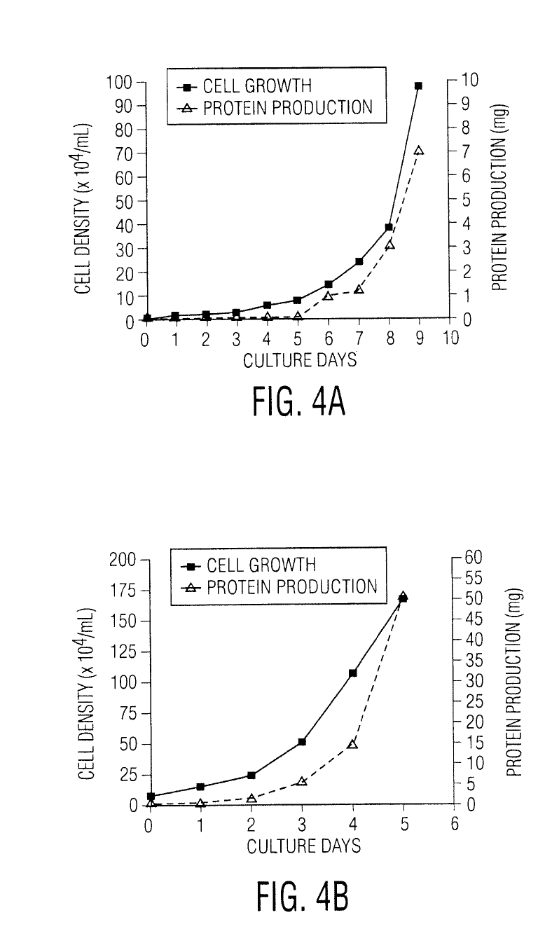

In order to determine the optimum temperature for culturing the cells in a bioreactor, the cells then were seeded at 5×10⁴ cells/ml in 5 liters of the CDPF medium (CD−CHO+Sup A) and incubated at a temperature of 34° C. or 36° C. for up to 9 days. (FIGS. 4A and 4B) in a bioreactor (Pilot Plant System; W350040-A Wheaton Science Products; 10 liter capacity). As shown in FIG. 4B, after 5 days, the cells that were incubated at 36° C. had a cell density of about 175×10⁴ cells ml, and produced about 50 mg of protein.

### Purification of Secreted Proteins

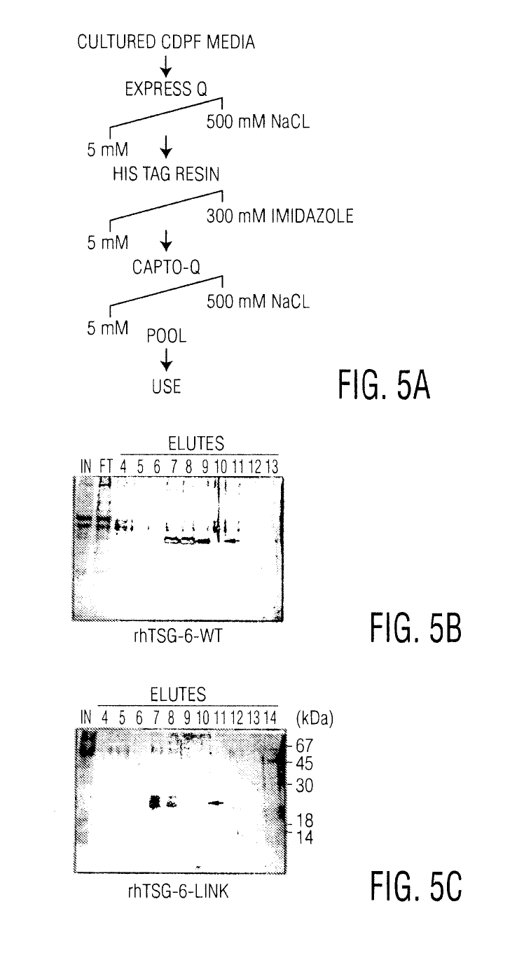

The more productive clones were suspended at 5×10⁴ cells/ml in 5 liters of the CDPF medium (CD−CHO+Sup A) hereinabove described in the bioreactor hereinabove described, for up to 8 days. The medium was clarified by centrifugation at 10,000 rpm for 10 min. Proteins were purified from the culture medium by sequential chromatography on an ion exchange column (300 mL resin bed; Express Ion Exchanger Q; Whatman/GE Healthcare, UK) eluted with 5 to 500 mM NaCl, and then a histidine binding nickel chelate column (25 mL resin bed; Ni-NTA agarose; Qiagen) eluted with 300 mM imidazole. The peak fractions were diluted 10-fold with 50 mM Tris-HCl (pH 7.4) and chromatographed on a second ion exchange column (10 mL resin bed; Capto Q; Pharmacia Biotech) eluted with 5 to 500 mM NaCl. (FIG. 5A). About 15 fractions were collected from each column, and subjected to SDS-PAGE. rhTSG-6 wild type (FIG. 5B) and rhTSG-6-LINK (FIG. 5C) were detected in the fractions. Multiple bands are detected with TSG-6-LINK (FIG. 5C) because of varying degrees of glycosylation.

The peak fractions from the last column either were frozen directly at −80° C. for storage or buffer exchanged by dialysis with 200 mM NaCl/50 mM Tris-HCl buffer before freezing.

### Bioassay of Recombinant Proteins in Chemically Injured Corneas

The experimental protocols were approved by the Institutional Animal Care and Use Committee of Texas A&M Health Science Center. Six-week-old male Lewis rats (LEW/Crl; Charles River Laboratories International, Inc.) weighing 180-200 g were used in all experiments. Rats were anesthetized by isoflurane inhalation. To create the chemical burn, 100% ethanol was applied to the whole cornea including the limbus for 15 seconds followed by rinsing with 10 ml of balanced salt solution. Then, the whole corneal and limbal epithelium was mechanically scraped using a surgical blade. Upon completion of the procedure, the eyelids of a rat were closed with one 8-0 silk suture at the lateral one third of the lid margin. At predetermined time points after injury, five rats each received injections of rh TSG-6 or rhTSG-6-LINK, each of which has a “His-tag” of six amino acid residues at the C-terminus (350 ng in 5 μL of PBS) obtained as hereinabove described, or the same volume of PBS was injected into the anterior chamber of the eyes of five rats. All injections were done with 32 gauge needle and syringe. Five uninjured (normal) rats served as controls.

After injury and treatment, the rat corneas were examined for corneal opacity and neovascularization under a dissecting microscope and photographed. Corneal opacity was assessed and graded by a blinded investigator who was an ophthalmologist as: grade 0, completely transparent cornea; grade 1, minimal corneal opacity, but iris clearly visible; grade 2, moderate corneal opacity, iris vessels still visible; grade 3, moderate corneal opacity, pupil margin but not iris vessels visible; and grade 4, complete corneal opacity, pupil not visible. For semi-quantitative estimate of neutrophil infiltration by assay for myeloperoxidase activity (MPO), the cornea was sectioned into small pieces and lysed in 150 μl of tissue extraction reagent containing protease inhibitors (Invitrogen). The supernatant was assayed for levels of pro-inflammatory cytokines and chemokines with commercial ELISA kits for IL-1β (Quantikine Kit; R & D Systems), and for MPO. (Rat MPO ELISA kit; HyCult biotech).

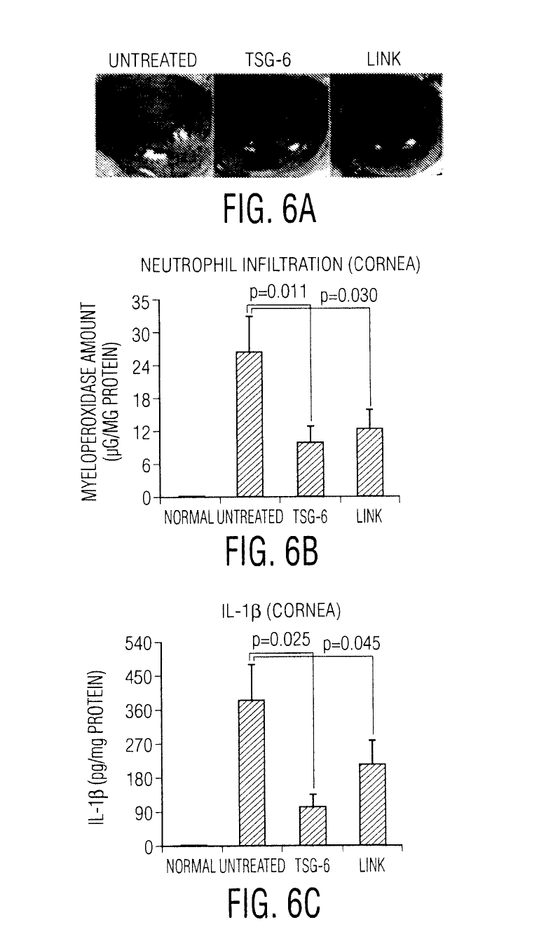

As shown in FIG. 6A, corneal opacity was reduced significantly in both rhTSG-6 and rhTSG-6-LINK-treated corneas. For an estimate of neutrophil infiltration, the concentration of myeloperoxidase (MPO) was assayed. Treatment with rhTSG-6 or rhTSG-6-LINK reduced the levels of MPO in the cornea significantly. (FIG. 6B). Also, the levels of the pro-inflammatory cytokine IL-1β were decreased significantly in the rhTSG-6 or rhTSG-6-LINK treated corneas as assayed by ELISA. (FIG. 6C).

The above results show that the rhTSG-6 and rhTSG-6-LINK proteins produced in accordance with the method of the present invention are effective in treating corneal injuries.

### Example 2

### Materials and Methods

hMSCs Culture

Frozen vials of hMSCs from bone marrow (Donor 7302R) were obtained from the Center for the Preparation and Distribution of Adult Stem Cells (formerly http://www.som.tulane.edu/gene_therapy/distribute.shtmLl; currently http://medicine.tamhsc.edu/irm/msc-distribution.html) that supplies standardized preparations of MSCs enriched for early progenitor cells to over 250 laboratories under the auspices of an NIH/NCRR grant (P40 RR 17447).

A frozen vial of about 10⁶ passage 1 hMSCs was thawed, and plated at 200 to 500 cells/cm² in 150 mm diameter plates with 30 mL of complete culture medium (CCM) that consisted of α-minimal essential medium (α-MEM; Invitrogen, Carlsbad, Calif.), 17% fetal bovine serum (FBS; lot-selected for rapid growth of hMSCs; Atlanta Biologicals, Inc, Norcross, Ga.), 100 units/mL penicillin, 100 μg/mL streptomycin, and 2 mM L-glutamine (Invitrogen). The cultures were incubated with replacement of medium every 2 days for 7 to 9 days until they were 70% confluent. The cultures were washed with PBS and the MSCs (passage 2) were harvested by incubation for 5 to 10 min at 37° C. with 0.25% trypsin and 1 mM EDTA.

### Plasmid Construction

Total RNA was isolated from hMSC cells that were stimulated to express TSG-6 by incubation of 3×10⁴ cells/cm² overnight with 10 ng/mL of TNF-α in CCM containing a reduced concentration of 2% FBS (Lee, et al., Cell Stem Cell, Vol. 5, pgs. 54-63 (2009)). About 1 μg of total RNA was used to produce the first strand cDNA pool by RT-PCR (Superscript II/oligo dT₁₂₋₁₈, Invitrogen). cDNAs encoding hTSG-6 (GenBank accession number: NM_{—}007115) were amplified by PCR using the following primers: 5′-CGGGGTACCATGATCATCTTAATTTACTT-3′ (sense), 5′-GGTGATCAGTGGCTA AATCTTCCA-3′ (antisense). The PCR products were sub-cloned into the Kpn I and Spe I sites in multi-cloning sites of a pEF4-Myc/His plasmid (cat. #V942-20; Invitrogen, Carlsbad, Calif.) and the plasmid was amplified in E. coli DH5α cells (cat. #18265-017; Invitrogen).

Synthesis of rhTSG-6-WT in Stably Transfected CHO Cells

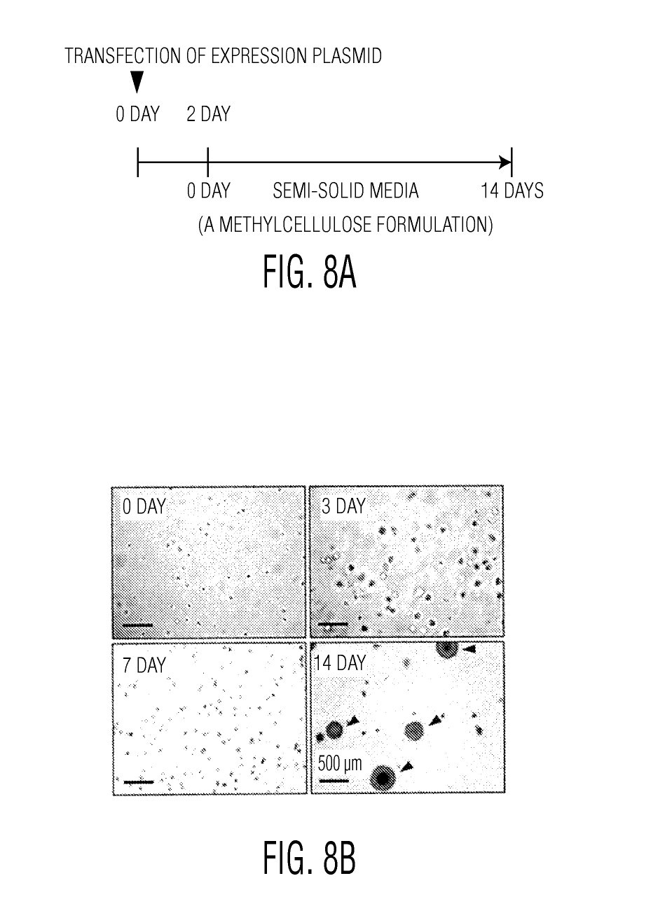

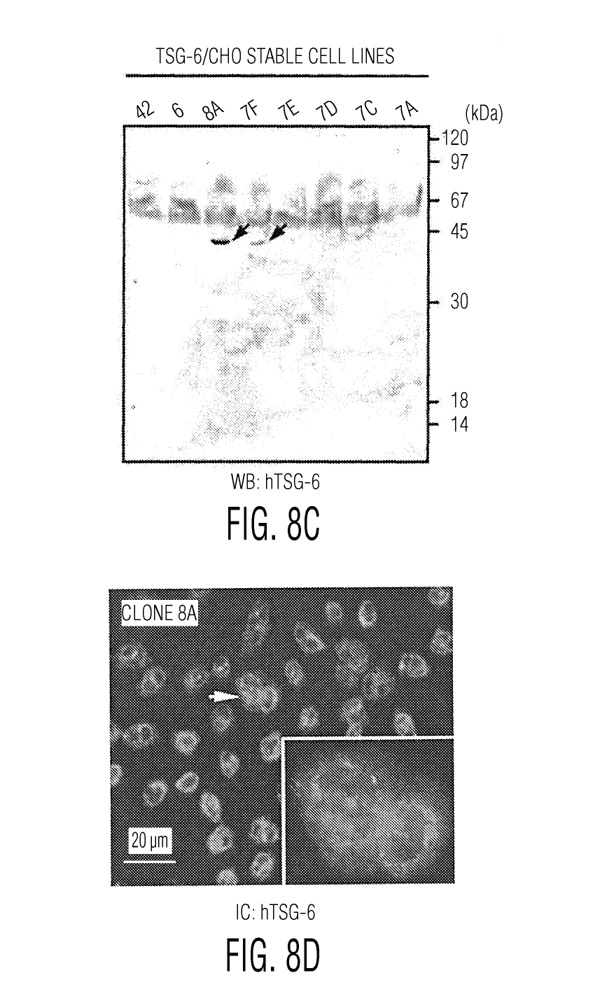

Chinese hamster ovary (CHO)-S cells (Invitrogen, Carlsbad, Calif.) were plated at 1×10⁵ cells in a 100 mm diameter culture dish with 10 mL IMDM (Iscove's Modified Dulbecco's Medium) containing 5% FBS (Premium Select; Atlantic Biologicals), 50 units/ml of penicillin, and 50 μg/mL of streptomycin. After incubation for 2 days, cells were transfected with 30 μg of the plasmid construct for expression of rhTSG-6-WT using 60 μl of lipofectamine reagent (Lipofectamine 2000™; Invitrogen) in serum-reduced medium (Opti-MEM; Invitrogen). Four hours later, the medium was replaced with 10 mL of 5% FBS/IMDM and the cells incubated further for another one day. The next day, the transfected cells were lifted and re-seeded in a 100 mm diameter culture dish with 9 mL of a methylcellulose based medium (ClonaCell-TCS medium, catalogue #03814; StemCell Technologies; http://www.stemcell.com/en/Products/Area-of-Interest/Semi-solid-cloning/ClonaCellHY-Medium-D-without-HAT.aspx) containing 500 μg/mL of Zeocin (Invitrogen) to select transformed clones with a rapid protocol (Jones, et al., J. Immunol., Vol. 171, pgs. 196-203 (2003); Kern, et al., Blood, Vol. 114, pgs. 3960-3967 (2009)). The samples were cultured 10-14 days to allow the clones to form spheres in the semi-solid selection media. The spheres were isolated using a pipette under a microscope. The spheres were expanded on 48 well plates in 5% FBS/IMDM containing 500 μg/mL of Zeocin until 70% confluence was achieved. At this point, we tested expression in the cells using immunocytochemistry with fluorescently labeled hTSG-6 antibodies (below). The secretion of proteins from each clone was tested by ELISA and selected clones were further tested by Western blotting of media. The most productive clones were expanded further in 24 well plates and then in 100 mm diameter culture dishes to achieve an adequate number of cells for storage in liquid nitrogen (FIG. 8.). After expansion, the clones were adapted sequentially to CD-CHO media.

### ELISA

For ELISA of secreted recombinant protein, a 96-well plate (Maxisorp; Nunc) was coated overnight at 4° C. with 100 μL/each well of 10 mg/ml monoclonal antibody specific for rhTSG-6 (clone A38.1.20; Santa Cruz Biotechnology, Inc.) in 0.2 M sodium bicarbonate buffer (pH 9.4). The plates were washed with PBS twice and blocked with 0.25% (wt/vol) BSA and 0.05% (vol/vol) Tween 20 in PBS for 30 min at room temperature. Plates were washed again with PBS. Samples of medium (100 μL) or standards of recombinant human TSG-6 protein (R&D Systems) in blocking buffer were added. After 2 hrs. at room temperature, the wells were washed with PBS followed by 50 μL/well of 0.5 mg/ml biotinylated anti-human TSG-6 (TSG-6 Biotinylated PAb Detection Antibody; R&D Systems). After 2 hrs. the plates were washed with PBS. Fifty microliters streptavidin-HRP (R&D Systems) were added to each well. The plates were covered and incubated for 20 min at room temperature. The plates were washed with PBS, 100 μl of substrate solutions (R&D Systems) was added, and the samples were incubated for 10 min at room temperature. Absorbance was read at 450 nm (Fluostar Optima; BMG Labtechnologies).

### Western Blots

Ten μL of each sample were separated by 12% SDS-polyacrylamide gel electrophoresis and blotted onto a polyvinylidene fluoride (PVDF) membrane. The blot was incubated with a primary antibody that reconginizes TSG-6 (200 μg/mL; clone A38.1.20; Santa Cruz Biotechnology, Inc.) for 1 hr at RT, and then with secondary antibody conjugated with horseradish peroxidase (1:4000, Santa Cruz) for 30 min. at RT. The gels were visualized with the ECL kit (Amersham Pharmacia).

### Immunocytochemistry

Cells at about 1×10⁴ cells/cm² were cultured on cover slips (12 mm diameter; catalogue #12-545-82; Fisher Scientific) in 24 well plates (catalogue #3524; Corning, N.Y.) in 5% FBS/IMDM for 3 days. The samples were fixed for 10 min in 4% paraformaldehyde (PFA), and washed with 1×PBS. The slides were blocked in 1×PBS containing 3% BSA and 0.2% Triton X-100 for 1 hr at RT. Cells were then incubated for 1 hr at RT in primary antibodies (200 μg/mL; TSG-6 clone A38.1.20; Santa Cruz Biotechnology, Inc. and 400 μg/mL; His(C-Term); Invitrogen) and washed three times with PBS. Cells were incubated with fluorescence-labeled secondary antibodies and nuclei were counter-stained with DAPI (10 μg/ml; Molecular Probes) for 30 min. at RT.

To stain aggregates, the aggregates were collected with cell lifter, transferred to a 50 mL conical tube, washed twice with PBS, and fixed with 4% PFA in PBS for 10 min. at room temperature. Then 1 mL of OCT solution (Sakura Finetek) was added to the cell aggregates and they were transferred into a histology mold. The mold was frozen in isopentane (Sigma) chilled by liquid nitrogen. Cryosections (about 10 μm) were prepared with a Microm HM560 cryostat. After blocking with 3% BSA and 0.2% Triton X-100 in 1×PBS for 1 hr. at room temperature, the samples were incubated overnight at 4° C. in primary antibodies (200 μg/mL; TSG-6 clone A38.1.20; Santa Cruz Biotechnology, Inc. and 10 μg/mL; HABP; Amsbio) and washed three times with PBS. The aggregates were incubated with fluorescence-labeled secondary antibodies and observed under a fluorescence microscope (Olympus) and digitized with a CCD camera. Images were optimized using Adobe Photoshop 7.0.

### Experiments in Spinner Cultures to Optimize the Medium

To optimize culture conditions, individual clones of transduced cells each were plated in two 150 mm diameter dishes at 3,000 cells/cm² in 30 mL of 5% FBS/IMDM containing 100 μg/mL of Zeocin. After 2 days, the cells were washed with PBS, lifted with 0.25% trypsin, and suspended in spinner culture bottles (Wheaton, Millville, N.J.) at 6×10⁴ cells/mL in 500 mL of chemically-defined and protein-free medium (CD CHO Medium, cat #10743-029; Invitrogen) without Zeocin. Trial experiments were carried out to test the effects of addition of a series of supplements by adding the following to the medium either separately or in combinations: (a) 11 mM of D-(+)-glucose (Sigma; G6152-100G); (b) 10 mL/L non-essential amino acid (MEM 100×; cat. #11140-050; Invitrogen); (c) 10 ml/L vitamin solution (MEM Vitamin Mixture 100×; cat. #11120-052; Invitrogen); (d) 10 ml/L lipid concentrate (Chemically defined, cat. #11905-031; Invitrogen); and (e) 10 ml/L of surfactant co-polymer (Pluronic F-68, cat. #24040-032; Invitrogen). The results made it possible to define an optimized chemically-defined and protein-free (OCDPF) medium for subsequent experiments.

### Preparation of CHO Clones for Culture in Spinner Cultures

Clones were plated in two 150 mm diameter dishes at 3,000 cells/cm² in OCDPF containing 100 μg/mL of Zeocin. After 2 days, the cells were washed with PBS and then were incubated in fresh OCDPF media for an additional 1 day. The medium was prepared with 1 liter CHO medium (CD-CHO cat. #10743-011; Invitrogen) that was supplemented with 10 mL hypoxanthine/thymidine supplement (HT 100×, cat. #11067-030; Invitrogen), 40 mL L-glutamine solution (final concentration 8 mM; L-Glutamine 200 mM; cat. #G6152-100G; Sigma), 2 grams D-(+)-glucose (cat. # G6152-100G; Sigma), 10 mL non-essential amino acids (cat. #11140-050; Invitrogen), 10 mL MEM vitamin solution (cat. #11120-052; Invitrogen). In addition, 4-methylumbelliferone sodium salt (M1508; SIGMA) was added to 50 μM concentration to decrease hyaluronan synthesis (Kakizaki et al., J. Biol. Chem., Vol. 279, pgs. 33281-33289 (2004)). Also, heparin (H4784, SIGMA) was added to 250 μg/mL concentration to promote suspension adaptation of TSG-6 stable cell lines and to increase the recovery rate of rhTSG-6 proteins (Li, et al., Molecular Biotechnology, Vol. 47, pgs. 9-17 (2011)). The cells were washed with PBS and lifted using trypsin. After centrifugation, the cells were re-seeded at about 6×10⁴/mL in 1 L of OCDPF medium for suspension culture in a spinner bottle and cultured further for 3 days.

### Bioreactor Culture of Stable Cell Line

For production of the proteins in a bioreactor, the cells that had been cultured in a spinner bottle for 3 days were suspended at 1×10⁵/mL in 5 liters of OCDPF medium, and incubated in a bioreactor (PILOT PLANT SYSTEM; W350040-A Wheaton Science Products; 10 liter capacity) that automatically titered the pH to 7.4 and monitored the oxygen content of the medium.

Purification of Secreted rhTSG-6

The culture medium was clarified by centrifugation at 17,000 g for 10 min. After brief sonication, the medium was passed through a 0.45 μm filter. The proteins were purified by a histidine binding nickel chelate column. In brief, the medium (5 L) was adjusted with an equilibration buffer and was loaded on the column (70 mL resin bed; Ni-Sepharose Excel; GE Healthcare) that had been equilibrated with the binding buffer (500 mM NaCl, 0.1% Triton X-100 and 2 M Urea in 20 mM phosphate buffer at pH 7.4). The column first was washed with 25 column volumes of an endotoxin removal buffer (500 mM NaCl, 0.025% Triton X-114, 0.1% Tween 20, 2 M Urea and 20 mM imidazole in 20 mM phosphate buffer at pH 7.4) (Reichelt, et al., Protein Expression and Purification, Vol. 46, pgs. 483-488 (2006)). In order to wash out any residual Triton-X114 and Tween 20, the column was washed with 40 column volumes of wash buffer (500 mM NaCl, 2 M Urea, and 10 mM imidazole in 20 mM phosphate buffer at pH 7.4) overnight. The recombinant protein then was recovered with about 400 mL of elution buffer (500 mM NaCl, 2 M Urea, and 500 mM imidazole in 20 mM phosphate buffer at pH 7.4) at a flow rate of about 3.5 mL/min. Fractions of 8 mL were collected and assayed by SDS-PAGE gel electrophoresis and TSG-6 ELISA. Thirty to thirty five fractions were pooled (around 300 mL). The sample was dialyzed against an equilibration Q buffer (50 mM NaCl and 2 M Urea in 50 mM Tris buffer) at pH 8.0 for application to the Q-Sepharose FF column (100 mL resin bed, GE Healthcare).

The dialyzed sample was loaded on the Q-Sephorose FF column, a strong anion exchanger that had been equilibrated with 10 column volumes of Q buffer. The column was washed with 20 column volumes of wash buffer (150 mM NaCl, 2 M Urea, and 50 mM Tris-HCl at pH 8.0). The bound proteins then were eluted with 3 column volumes of elution buffer (400 mM NaCl, 2 M Urea, 50 mM Tris-HCl at pH 8.0). The peak fractions were pooled, dialyzed against PBS, D-(+)-Trehalose (T9531, SIGMA) was added to 5% final concentration, and the sample then was frozen at −80° C. for storage.

### Assays of Protein and Endotoxin

The protein content of the samples was assayed with the Bradford method (Quick Start Bradford Protein Assay; Bio-Rad). Endotoxin was assayed with the Limulus amoebocyte lysate chromogenic assay (QCL-1000™ Endpoint Chromogenic LAL Assays, Lonza) according to the manufacturer's instructions. Briefly, duplicates of 50 μl of each test sample, 4 standards, and a negative control (apyrogenic LAL water) were transferred to endotoxin free tubes. The samples were incubated at 37° C. for an initial period of 10 min., for 10 min. after addition of 50 μl LAL lysate, and then for 8 min. after mixing gently in 50 μl of substrate. The reaction was terminated with 100 μl of stop reagent. The samples of 200 μl of were transferred to a microtiter plate and within 30 min. the absorbance at 405 to 410 nm was recorded.

Deglycosylation of rhTSG-6

N-Glycocidase A (5 mU, Roche Life Science) was diluted with 100 mM sodium acetate buffer (pH 5.0) to a concentration of 0.25 mU per 16 μl. rhTSG-6 (1.2 μg) was added, the sample volume was increased to 20 μL with the enzyme diluent, and it was incubated at 37° C. for 15, 60, and 180 min. Half of the reaction solution was separated by 10% SDS-polyacrylamide electrophoresis and stained by Coomassie Brilliant Blue solution. The remainder was separated by 10% SDS-polyacrylamide gel electrophoresis and transferred to a nitrocellulose (NC) membrane for staining glycoproteins (Pierce Glycoprotein Staining Kit; #24562; Thermo Scientific). In brief, the NC membrane was washed with 10 mL 3% acetic acid for 10 min. and then transferred to 10 mL of oxidizing solution. After 15 min. gentle agitation, the membrane was washed with 10 mL of 3% acetic acid solution for 5 min. three times. The membrane was soaked in 10 mL of Glycoprotein Staining reagent for 15 min. and then transferred to 10 mL of Reducing Solution and washed gently with 3% acetic acid for 5 min. three times. To visualize the stained bands, the membrane was washed with ultrapure water. The stained membrane as magenta color was kept in 3% acetic acid solution.

Half-Lives of rhTSG-6 in Mice

Male C57BL/6 mice 7-8 weeks old (20-22 g) were purchased from Jackson Laboratory. About 50 μg of myeloma cell-extracted rhTSG-6 (R&D systems, 2104-TS) or CHO cell-derived rhTSG-6 were injected into the tail vein. Blood samples were collected by cardiac puncture at 0.5 min., 30 min., 1 h, 3 h, 6 h, 12 h, and 24 h after rhTSG-6 injection. To separate the plasma, the mice were anesthetized with ketamine/xylazine, the blood was recovered in heparin coated capillary blood collection tubes (Terumo), and it was centrifuged at 3000 g for 10 min. at 4° C. The centrifugation step was repeated twice to minimize platelet contamination and the clear plasma fraction was stored at −80° C. rhTSG-6 protein levels in plasma were determined by ELISA. The data were fitted to two compartment models and the half-lives of distribution (t_{1/2α}) and elimination (t_{1/2β}) were calculated by non-linear least squares regression using GraphPad Prism. (myeloma-derived TSG-6: t_{1/2α}=0.15 hr. t_{1/2β}=0.20 hr. CHO cell-derived TSG-6: t_{1/2α}═0.08 hr. t_{1/2β}=0.47 hr.)

### LPS-Induced Inflammation Model

Mice were randomized, to, receive intravenously either PBS or rhTSG-6 (25 μg or 50 μg) mixed with 60 μg of LPS from Escherichia coli 055:B5 (Sigma, L2880) to induce inflammation. After 6 hours, the mice were euthanized by a lethal dose of 5% isoflurane in 100% oxygen followed by cervical dislocation. Spleens were collected and frozen in dry ice and stored at −80° C. before analysis. RNA was extracted from spleen (RNeasy Mini Kit; QIAGEN). Approximately 1 μg of total RNA was used to synthesize double-stranded complementary DNA by reverse transcription (Super Script III, Life Technologies). The complementary DNA was analyzed by real-time PCR (ABI7900 Sequence Detector, Applied Biosystems). For assays for mouse-specific transcripts, mouse-specific primers and probes (Life Technologies) were used: IL-6 (Mm00439653_m1) and IFN-γ (Mm00599890_m1). For relative quantitation of gene expression, mouse-specific GAPDH primer and probes (Mm99999915_g1) were used.

### Statistical Analysis

Comparisons of parameters among the groups were made by one-way ANOVA using SPSS software (version 12.0). Differences were considered significant at P<0.05.

### Results

Preparation of Stable Lines of CHO Cells Expressing rhTSG-6.

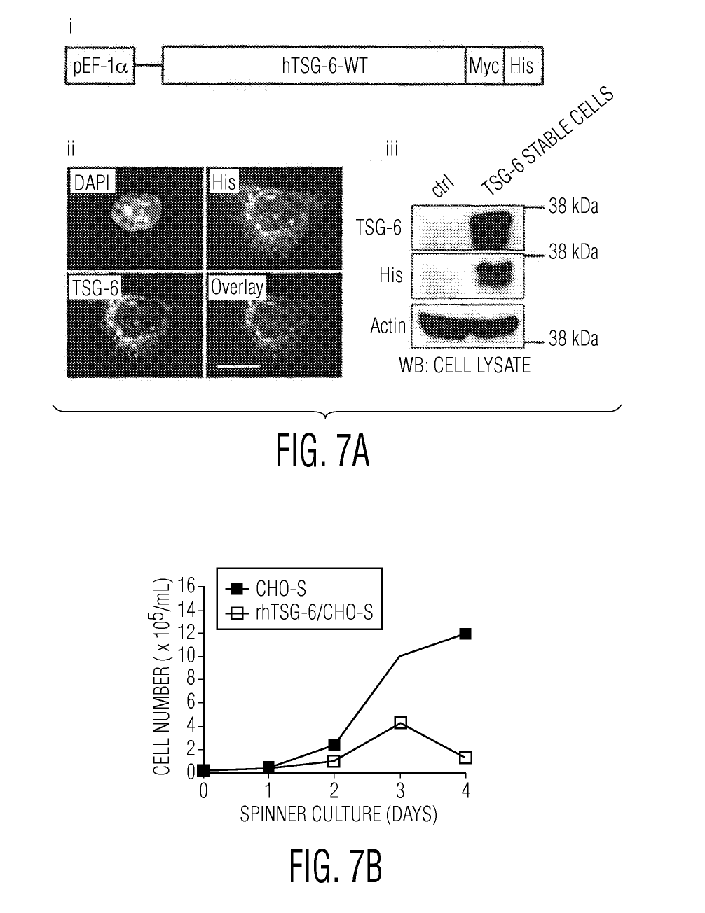

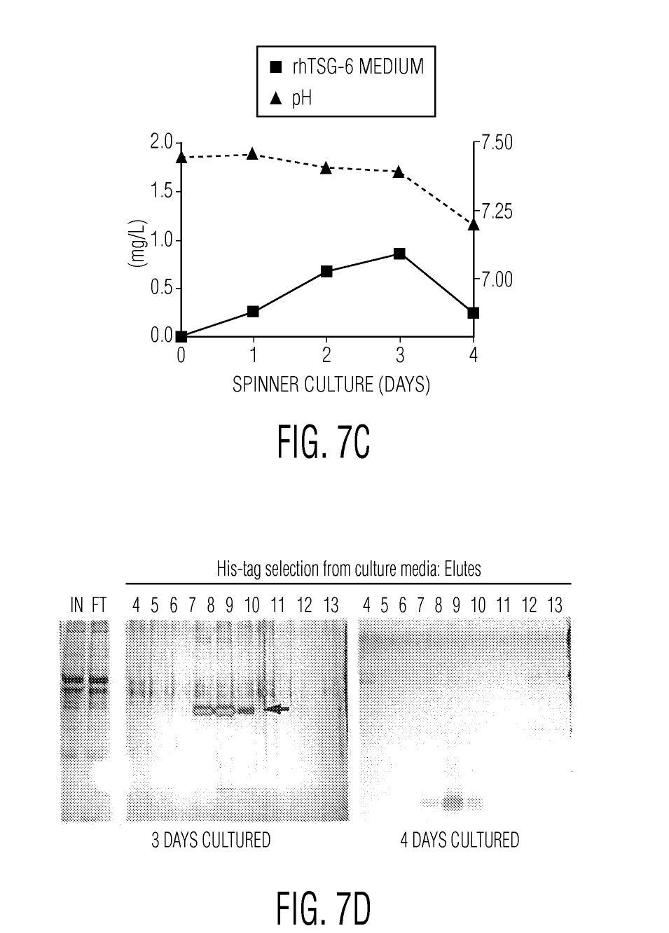

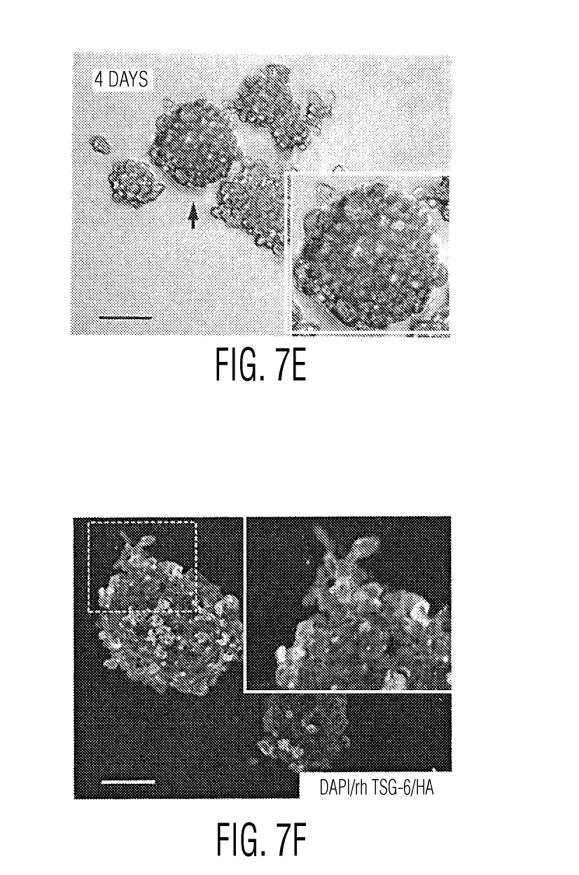

A DNA construct containing sequences for human wild type TSG-6 (Day, 1996) was prepared from RNA extracted from human MSCs incubated with TNF-α to increase expression of TSG-6 (Lee, 2009). The sequences were cloned into a plasmid with a promoter of elongation factor EF-1α to avoid the silencing occasionally encountered with other promoters (FIG. 7A, i). The plasmid also incorporated Myc-tag and His-tag sequences at the C-terminus to facilitate detection and purification of the proteins. After the cDNAs were sub-cloned into the plasmid (FIG. 7A, i), the plasmid was amplified in E. coli, and the insert was sequenced to verify its structure (not shown). An expression plasmid then was used to prepare stable transfectants of CHO cells using a lipofectamine protocol. Expression of rhTSG-6 was confirmed by Western blot and immunostaining for rhTSG-6 and the C-terminal His-tag after transient transfection in CHO-S cells (FIG. 7A, ii, iii). For isolation of clones, the cells were cultured on a methylcellulose medium containing about 800 μg/mL of Zeocin that makes it possible to isolate clones in 2 to 3 weeks (Jones, et al., J. Immunol., Vol. 171, pgs. 196-203 (2003); Kern, et al., Vol. 114, pgs. 3960-3967 (2009). To identify clones that secreted the recombinant protein, medium from the clones was assayed for expression by Western blotting and the results confirmed by immunocytochemistry of cultures of the clones (FIG. 8).

The most highly expressing clones synthesized rhTSG-6 at a rate that was about 100-fold greater than the rate previously obtained with human MSCs that were shown to have secreted relatively large amounts of rhTSG-6 after they were activated by incubation with TNF-α (Lee, 2009), i.e. about 500 ftmoles of rhTSG-6/10⁶ CHO cells/24 hr versus 5 ftmoles by human MSCs incubated with TNF-α.

### Optimization of Conditions for Production of TSG-6 in a Spinner Culture Bottle

For production of the recombinant protein, the expanded CHO clones first were incubated in a spinner culture flask with a commercially available chemically-defined and protein-free medium for CHO cells (CD-CHO Medium; Invitrogen). As indicated in FIG. 7B, the transduced cells expanded but at a slower rate than untransduced CHO-S cells. Surprisingly, however, the yield of the transduced cells decreased sharply between day 3 and day 4. Also, there was a sharp decrease in the amount of rhTSG-6 recovered from the medium (FIGS. 7C and D). Microscopy of the cultures demonstrated that between days 3 and 4, the CHO formed large clusters of cells (FIG. 7E) that contained both rhTSG-6 and hyaluronan (FIG. 7F). The decrease in yield of both cells and rhTSG-6 after day 3 therefore was explained by the well-documented tendency of the protein to bind hyaluronan (Baranova, J. Biol. Chem., Vol. 288, pgs. 29642-29653 (2013)) and the fact that the CHO cells, like many cells in culture, are surrounded by a brush border of hyaluronan (Evanko, et al., Arteriosclerosis, Thrombosis, and Vascular Biology, Vol. 19, pgs. 1004-1013 (1999)). The affinity of TSG-6 increases at acidic pHs with a peak at about pH 6 (Heng, et al., J. Biol. Chem., Vol. 283, pgs. 32294-32301 (2008); Higman, et al., J. Biol. Chem., Vol. 289, pgs. 5619-5634 (2014)). Therefore the tendency of the rhTSG-6 to aggregate CHO cells in the spinner cultures was enhanced by the decrease in pH (FIG. 7C) that probably reflected decreased gaseous exchange and an accumulation of CO₂ in the medium (Velez-Suberbie, et al., Biotechnology Progress, Vol. 29, pgs. 116-126 (2013)).

### Conditions for Decreasing Aggregation of CHO Cells.

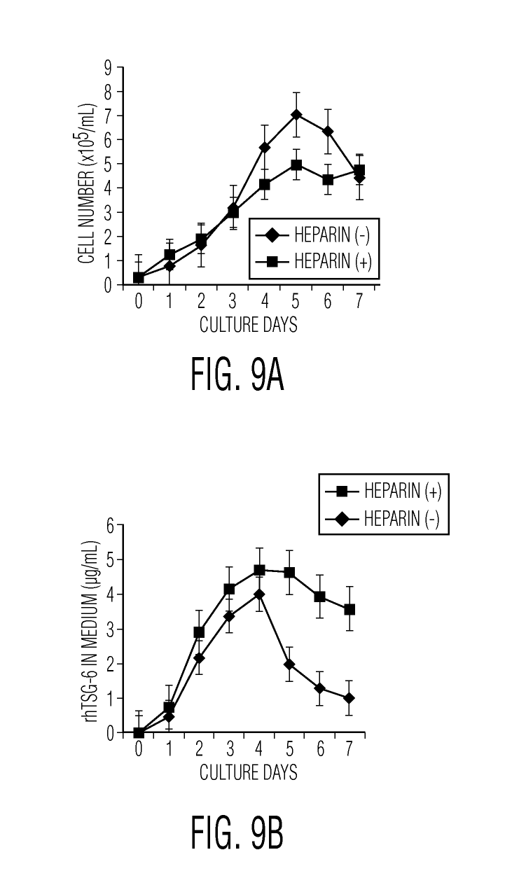

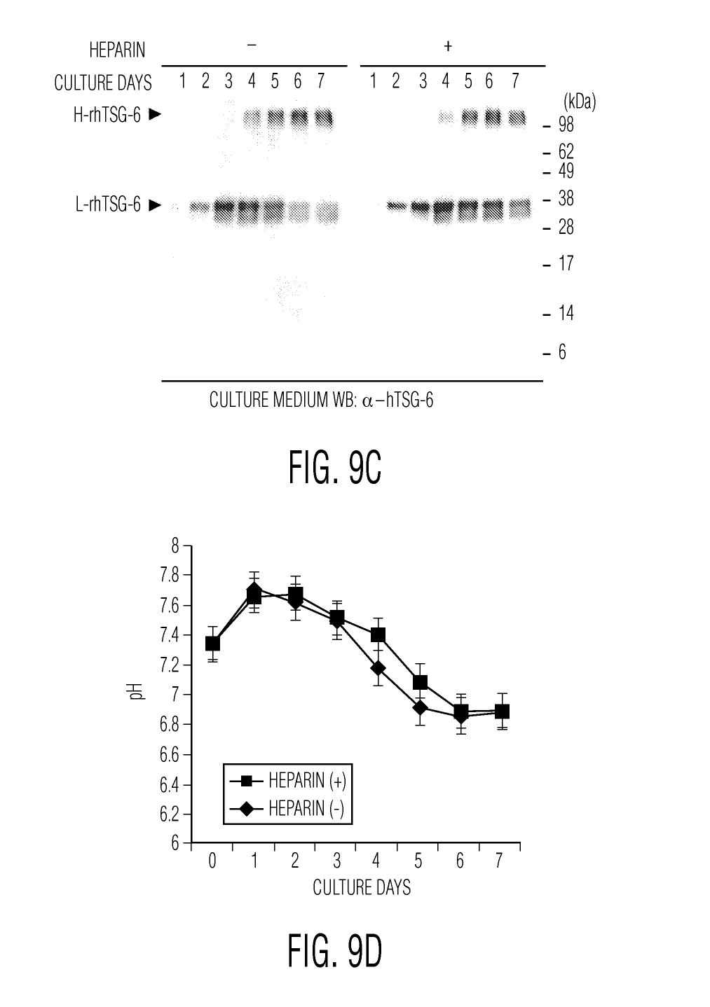

To reduce the tendency of the rhTSG-6 to cause aggregation of the CHO cells, we instituted two measures. One was addition to the medium an inhibitor of hyaluronan synthesis, methylumbelliferone (Kakizaki, 2004). The second measure was to add heparin to the medium to compete with the binding of rhTSG-6 to hyaluronan (Table 1). The addition of heparin to the spinner cultures improved the decrease with time in culture of both cell number (FIG. 9A) and production of rhTSG-6 (FIG. 9B)(Li, 2011). It also increased slightly the amount of protein that was recovered in a monomeric form and correspondingly decreased the aggregated form (FIG. 9C). But addition of heparin to spinner culture did not prevent the decrease in pH (FIG. 9D), an observation suggesting that better control of pH would be helpful.

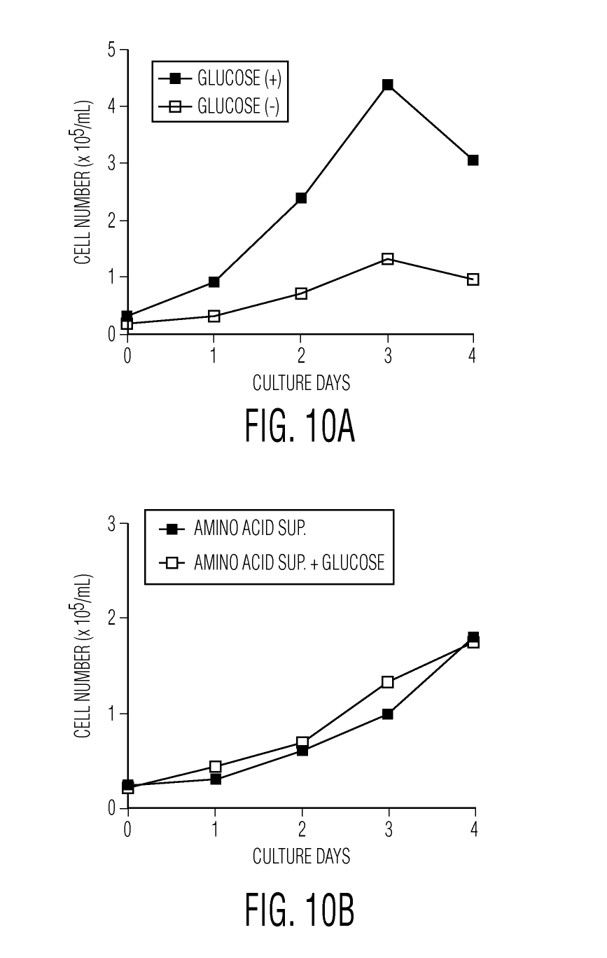

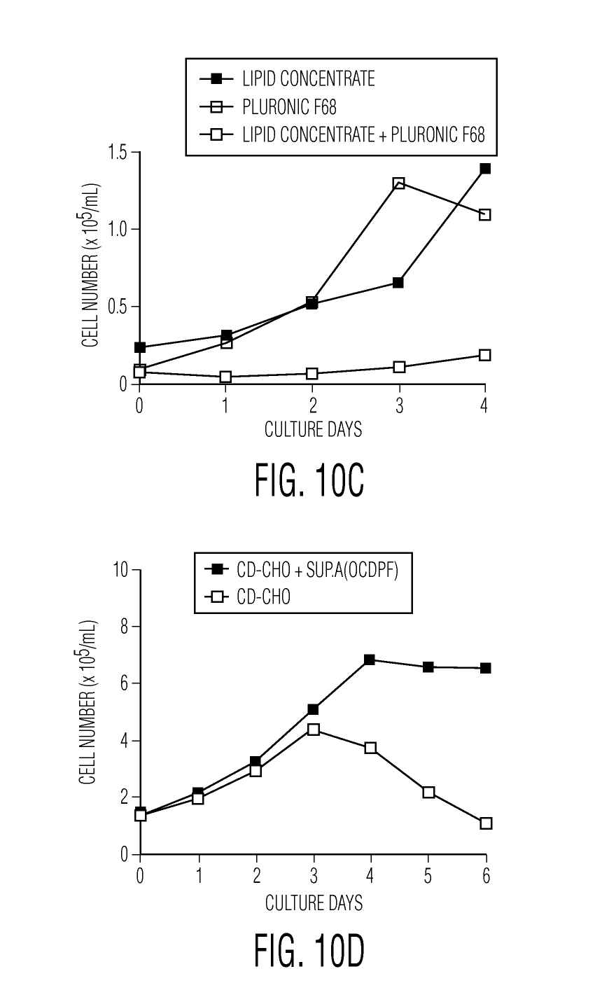

Several additional conditions to improve the yield also were instituted. The medium was supplemented with hypoxanthine/thymidine (Table 1) as suggested by Chen et al., Journal of Bioscience and Bioengineering, Vol. 114, pgs. 347-352 (2012) to increase the yield of cells and recombinant proteins (not shown). The medium was supplemented further with a high level of glucose (Table 1) to improve cell yields (FIG. 10A). Separate additions of a lipid concentrate or a surfactant polymer (Pluronic F68) also improved cell yield but, surprisingly, a combination of the two inhibited the system (FIG. 10C). Therefore these supplements were omitted. Supplementation with non-essential amino acids (Table 1) protected against the decrease in cell yield between days 3 and 4 (compare FIGS. 10A and B). The results of the experiments provided a medium (OCDPF) that is defined in Table 1 below.

TABLE 1
Composition of optimized chemically defined protein free medium
(OCDPF medium).

| Supplements added to 970 ml | | | |
|---|---|---|---|
| CD-CHO | | | Company (cat. #) |
| HT Supplement | 10 | ml | Invitrogen (11067-030) |
| D-(+)-glucose | 2 | g | Sigma (G6152) |
| MEM non-essential amino acid | 10 | ml | Invitrogen (11140-050) |
| MEM vitamin solution | 10 | ml | Invitrogen (11120-052) |
| 1M Methylumbelliferone sodium salt | 50 | μl | Sigma (M1508) |
| Heparin sodium salt | 250 | mg | Sigma (H4784) |

### Scalable Production in a Bioreactor.

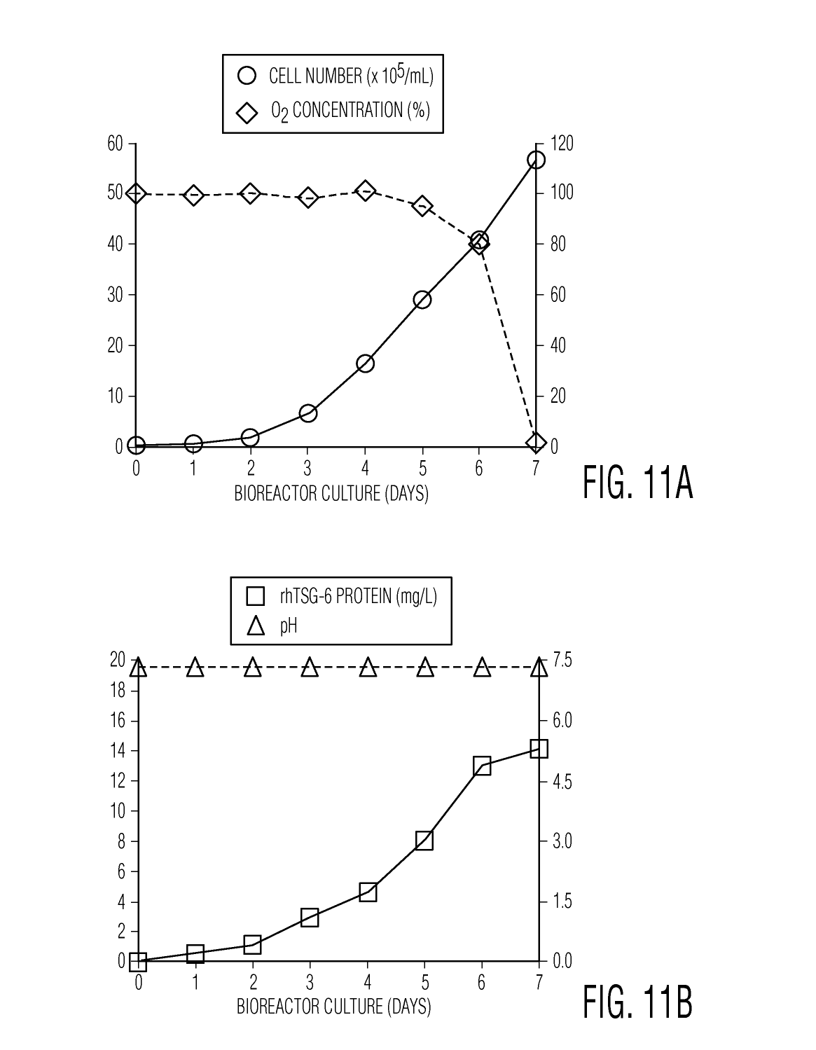

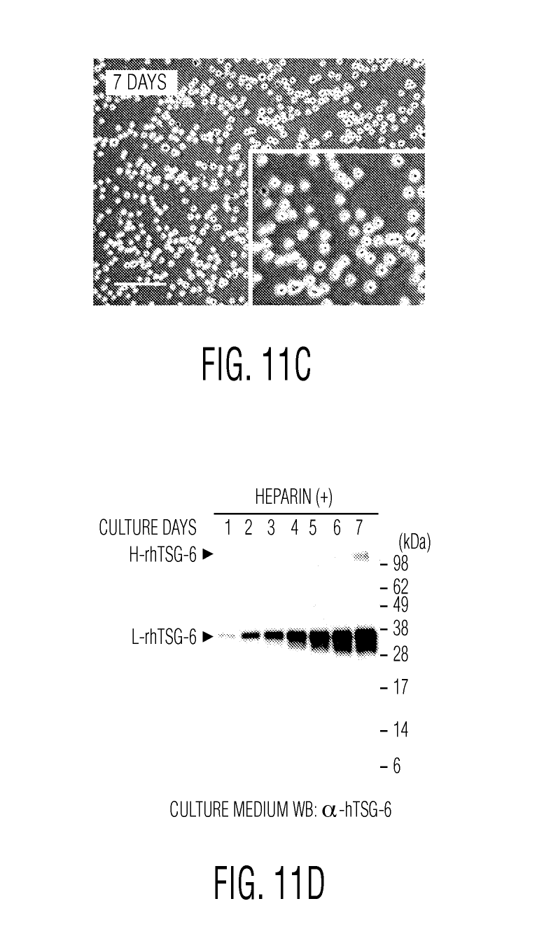

In addition to the above measures, the spinner culture system was replaced with a bioreactor that allowed control of pH and oxygen. Under the optimized conditions in the bioreactor, the cell number was increased almost 10-fold (from about 7×10⁵ in FIG. 9A to about 60×10⁵ per mL in FIG. 11A). The bioreactor, however; did not provide complete control of the incubation system, because the oxygen concentration fell drastically on day 7 (FIG. 11A), apparently because of the frequently encountered problem of insufficient gaseous exchange as the cell concentration increased (Velez-Suberbie, et al., Biotechnology Progress, Vol. 29, pgs. 116-126 (2013)). The bioreactor did control pH (FIG. 11B). Most importantly, the yield of TSG-6 in the medium increased to 10 to 14 mg/liter (FIG. 11B). In addition, the CHO cells did not aggregate (FIG. 11C) and most of the TSG-6 in the medium was recovered in a monomeric form (FIG. 11D).

Purification of the rhTSG-6

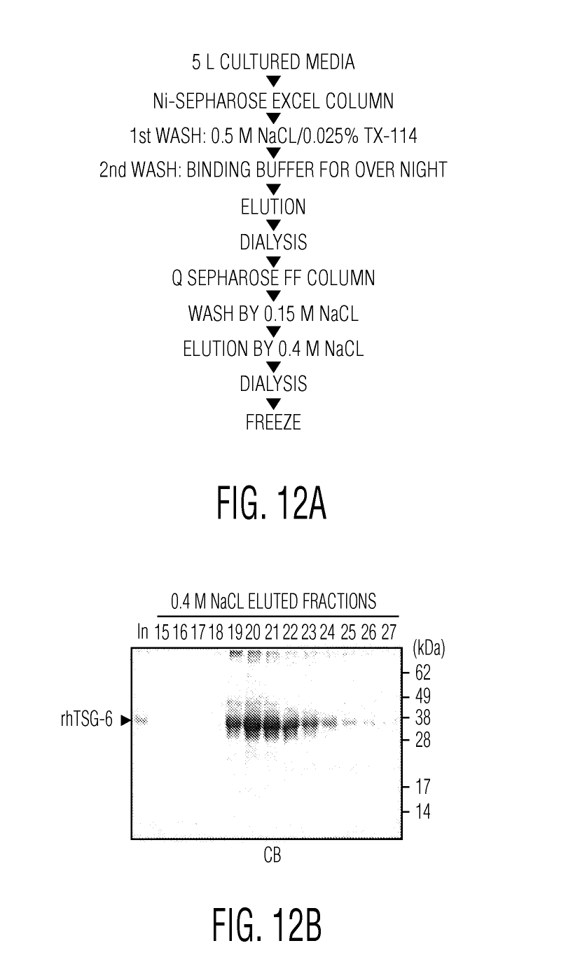

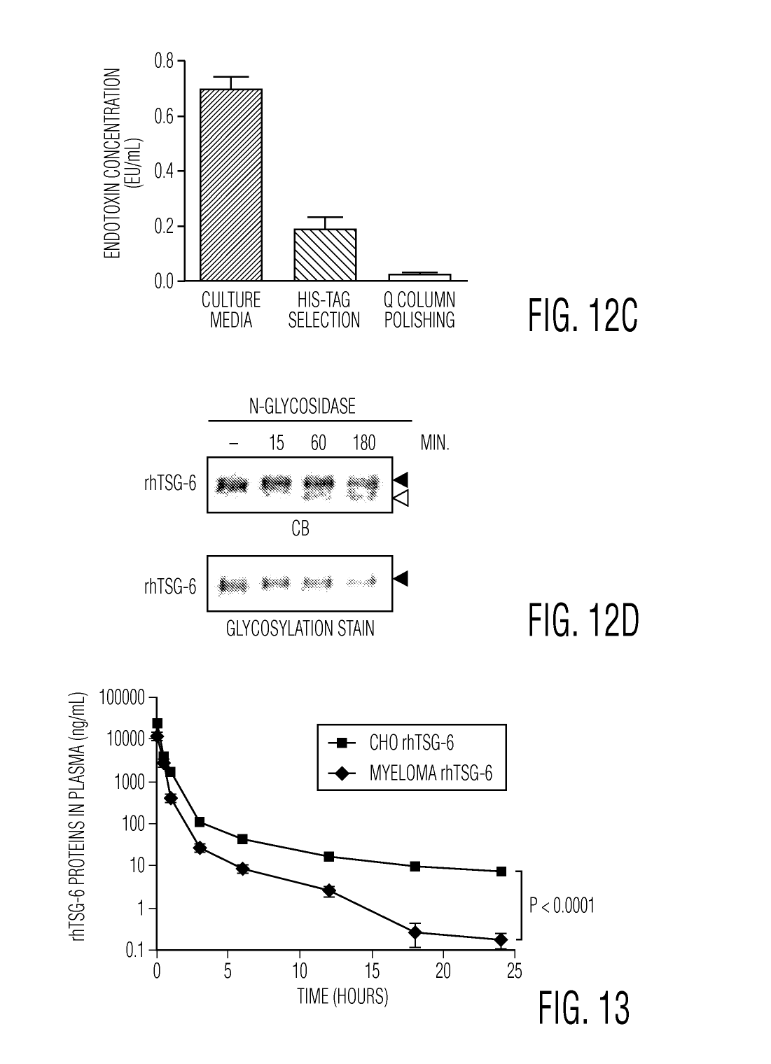

In initial experiments to purify the rhTSG-6, it was observed that the protein tended to self-aggregate in an apparently irreversible manner at 4° C. Therefore the culture medium was stored at −20° C., but after thawing, the protein was purified at room temperature. As a first step in purification, the culture medium was chromatographed on a Ni-chelate column (FIG. 12A) to take advantage of the His-tag engineered into the C-terminus of the protein (FIG. 7A). After binding of the protein, the column was washed with Triton X-114, a step shown to remove endotoxin (Buetler, Molecular Nutrition and Food Research, Vol. 55, pgs. 291-299 (2011)). The rhTSG-6 then was eluted and the protein chromatographed on an anion exchange column. The isolated protein was homogeneous as assayed by stained electrophoretic gels and primarily in a monomeric form (FIG. 12B). Also, the resulting protein was shown to be largely free of endotoxin (FIG. 12C). A concentration of 0.025% was used for the final protocol to provide purified rhTSG-6 with 0.375˜0.625 EU/mg of rhTSG-6 without sacrificing the recovery of rhTSG-6. The FDA threshold for the pyrogenic human dose is 5 E.U. per kg (Malzala, et al., Journal of Pharmaceutical Sciences, Vol. 97, pgs. 2041-2044 (2008)) and therefore would permit a dose of up to 7 mg/kg of the purified rhTSG-6.

In addition, digestion with N-glycosidase indicated that it was glycosylated (FIG. 12D). The purification of the rhTSG-6 is summarized in Table 2 below. Of interest was that the rhTSG-6 accounted for 18 to 20% of the total protein in the medium. A 4- to 5-fold purification was obtained with chromatography on the Ni-chelate column with a recovery of about 50%. The polishing step on the anion exchange column provided a higher yield and the overall yield from the culture medium was about 45%. After elution from the anion exchange column, the protein was dialyzed against PBS, trehalose was added to a final concentration of 5%, and it was stored at −20° C. Samples stored at −20° C. for up to 3 months and then thawed were monomeric by gel electrophoresis (as in FIG. 12C) and were active in suppressing inflammation in vivo in the LPS mouse model (see below).

TABLE 2
Synthesis and Purification of rhTSG-6 in Bioreactor.

| | | | | Over-all | Ratio of |
|---|---|---|---|---|---|
| | | | | recovery | rhTSG-6/ |
| | Volume | Protein | rhTSG-6 | of | Proteins |
| Sample | (mL) | (mg) | (mg) | rhTSG-6 | X 100 |
| ^{a}OCDPF culture | 5,000 | 309.6 | 60 | — | 18-20% |
| medium | | | | | |
| Ni-Sepharose | 297.5 | 35.4 | 32.2 | 54% | 81 to 99% |
| Excel eluate | | | | | |
| Q Sepharose | 56 | 27.4 | 27.3 | 46% | 99% |
| eluate | | | | | |

^{a}Optimized chemically-defined protein free medium with supplements indicated in Table 1.

Metabolic Half-Life of the rhTSG-6

One measure of the biological activity of a secreted glycoprotein like TSG-6 is its metabolic half-life in vivo, because misfolded and unglycosylated proteins tend to be cleared rapidly (Sinclair, et al., Journal of Pharmaceutical Sciences, Vol. 94, pgs. 1626-1635 (2005)). Therefore 50 μg of the protein were injected intravenously into C57BL/6 mice and the metabolic half-life in plasma was assayed. As indicated in FIG. 13, the initial distribution phase of the rhTSG-6 synthesized with the system described here was about the same as for commercially available rhTSG-6 synthesized by mouse myeloma cells and extracted from the cells (CHO-rhTSG-6: 0.08 hr. vs. myeloma-rhTSG-6: 0.15 hr.). The elimination phase, however, was over twice as long for the rTSG-6 isolated here from CHO cells (CHO-rhTSG-6: 0.47 hr. vs. myeloma-rhTSG-6: 0.20 hr.). The difference probably is explained by the fact that the rhTSG-6 from the CHO cells is a secreted glycosylated form of the protein whereas the rhTSG-6 from the melanoma cells is purified from cell lysates.

### Suppression of Inflammation In Vivo

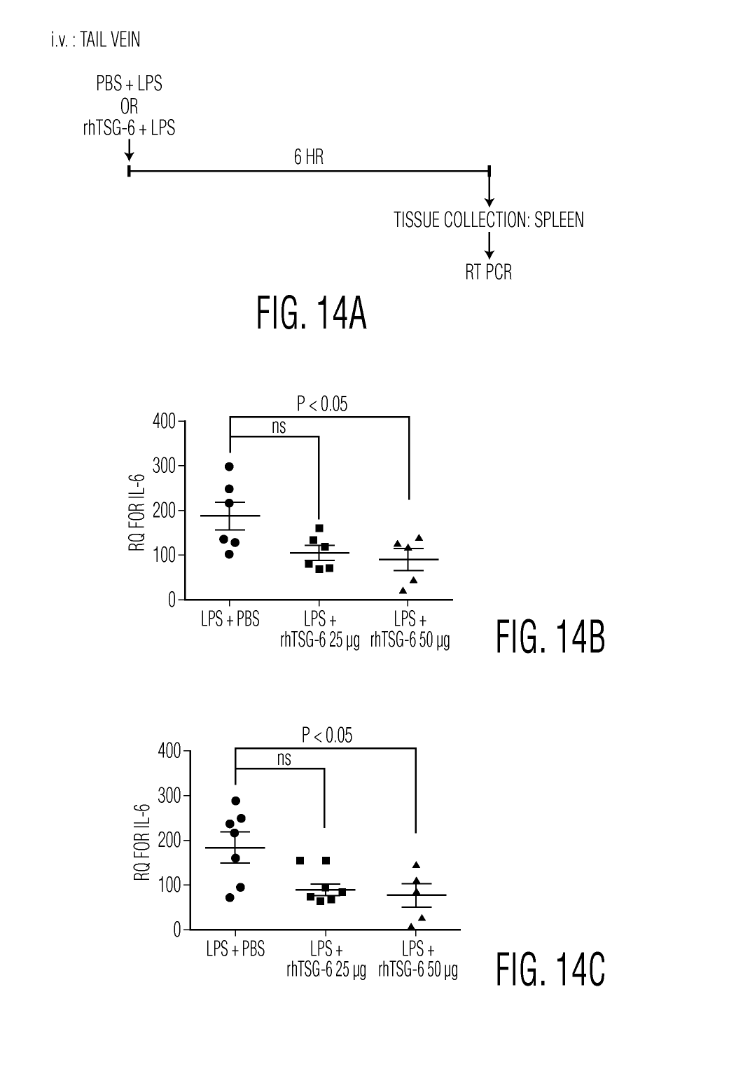

TSG-6 was shown to suppress inflammation in a series of different animal models (Prockop, et al., Molecular Therapy, Vol. 20, pgs. 14-20 (2012)). Most of the models require some technical expertise. Therefore, the rhTSG-6 was tested in a simple model in which inflammation is produced in mice by intravenous injection of the bacterial extract LPS and RT-PCR assays are used to detect the increase in expression of pro-inflammatory cytokines in the spleen. As indicated in FIG. 14, intravenous administration of 50 μg of rhTSG-6 from CHO cells suppressed the LPS-induced mRNA levels for both IL-6 and IFNγ in spleen (p<0.05). The results suggested the effect was dose dependent because 25 μg of rhTSG-6 did not produce a significant change.

### DISCUSSION

Synthesis of rhTSG-6 has been an unmet challenge since the protein first was discovered over 20 years ago (Wisniewski, (2004). Limited amounts were produced in several systems, but scalable production was not achieved. The results presented here demonstrate that cellular synthesis of rhTSG-6 is limited by well-known characteristics of the protein. It belongs to a family of hyaluronan binding proteins, termed hyadherins, that interact extensively with proteins in the extracellular matrix (Higman, 2014). As part of its interaction with matrix components, TSG-6 forms cross-links in hyaluronan either by binding directly to the linear high molecular weight glycosoaminoglycan or by serving as a cofactor and catalyst for the covalent transfer of heavy chains from inter-α-inhibitor to hyaluronan (Baranova, 2013). As demonstrated here, these properties limit production of the recombinant protein by CHO cells because the rhTSG-6 bound to and probably cross-linked the hyaluronan that forms a brush found on most cultured cells (Evanko, 1999). As a result; the CHO cells formed large aggregates which limited both the propagation of the cells and the amount of rhTSG-6 recovered from the medium. In addition, the tendency of the protein to self-aggregate presented a serious technical challenge.

The protocol developed here largely surmounts these problems. The dramatic tendency of the CHO cells to be aggregated by the newly-synthesized rhTSG-6 was reduced by use of an inhibitor of hyaluronan synthesis and addition of heparin as a competitor for the binding of TSG-6 to hyaluronan. Optimization of a standard medium for culture of CHO cells increased production and provided conditions under which the secreted rhTSG-6 accounted for 18 to 20% of the total protein. To prevent aggregation, procedures to concentrate the medium were avoided and the purification steps were performed at room temperature.

The yields of rhTSG-6 obtained here in a laboratory scale bioreactor of 5 liters should be adequate to allow, for the first time, structure studies on TSG-6 similar to the structural studies performed with the N-terminal half of the protein that contained the hyaluronan binding domain and that was synthesized in bacteria (Day, 1996). It also should be adequate for extensive studies in mouse and rat models for human diseases. The amounts also may be adequate for local administration in larger animals and human subjects for conditions such as corneal defects and joint injuries or diseases. Still larger amounts probably will be required for systemic administration in large animals and human subjects, but the protocol was designed to be scalable for production in large bioreactors and purification with chromatographic systems that can be enlarged readily. Therefore it should be feasible to overcome the problems of scale that limited earlier attempts to use the protein for clinical therapies. Initial experiments in mouse models suggested that the protein would be useful to treat arthritis (Giant, et al., Arthritis and Rheumatism, Vol. 46, pgs. 2207-2218 (2002); Mindrescu, et al., Arthritis and Rheumatism, Vol. 46, pgs. 2453-2464 (2002); Bardos, et al., Am. J. Pathology, Vol. 159, pgs. 1711-1721 (2001); Mindrescu, et al., Arthritis and Rheumatism, Vol. 43, pgs. 2668-2677 (2000)). More recent observations raise the possibility that it may have wide applications in suppressing excessive inflammation in a large number of diseases (Prockop, 2012).

The disclosures of all patents, publications (including published patent applications), depository accession numbers, and database accession numbers are incorporated herein by reference to the same extent as if each patent, publication, depository accession number, and database accession number were specifically and individually incorporated by reference.

It is to be understood, however, that the scope of the present invention is not to be limited to the specific embodiments described above. The invention may be practiced other than as particularly described and still be within the scope of the accompanying claims.

What is claimed is:

1. A method of producing a biologically active protein or polypeptide, or biologically active fragment, derivative or analogue thereof, comprising:
(a) introducing into mammalian cells a polynucleotide encoding a biologically active protein or polypeptide or a biologically active fragment, derivative, or analogue thereof;
(b) culturing said cells by suspending said cells in a protein-free medium, wherein said medium includes at least one agent that suppresses production of hyaluronic acid or hyaluronan or a salt thereof by said cells, wherein said cells are cultured for a time sufficient to express said biologically active protein or polypeptide, or a biologically active fragment, derivative, or analogue thereof; and
(c) recovering said expressed biologically active protein or polypeptide, or a biologically active fragment, derivative, or analogue thereof from said cells.

2. The method of claim 1 wherein said mammalian cells are CHO cells.

3. The method of claim 1 wherein said biologically active protein or polypeptide is TSG-6 protein or a biologically active fragment, derivative, or analogue thereof.

4. The method of claim 3 wherein said TSG-6 protein or biologically active fragment, derivative, or analogue thereof has at least one histidine residue at the C-terminal thereof.

5. The method of claim 4 wherein said TSG-6 protein or biologically active fragment, derivative, or analogue thereof has 6 histidine residues at the C-terminal thereof.

6. The method of claim 1 wherein said at least one agent that suppresses production of hyaluronic acid or hyaluronan or a salt thereof by said cells is 4-methylumbelliferone.

7. The method of claim 1 wherein said medium further includes at least one agent that inhibits or prevents the aggregation of said cells.

8. The method of claim 7 wherein said at least one agent that inhibits or prevents the aggregation of said cells is selected from the group consisting of heparin, dextran sulfate, ferric citrate, and combinations thereof.

9. The method of claim 8 wherein said at least one agent that inhibits or prevents the aggregation of said cells is heparin.

10. A biologically active protein or polypeptide, or biologically active fragment, derivative, or analogue thereof produced by the method of claim 1.

11. A composition comprising:
(a) the biologically active protein or polypeptide, or biologically active fragment, derivative, or analogue thereof of claim 10; and
(b) an acceptable pharmaceutical carrier.

12. A method of producing a biologically active protein or polypeptide, or biologically active fragment, derivative, or analogue thereof, comprising:
(a) introducing into mammalian cells a polynucleotide encoding a biologically active protein or polypeptide, or a biologically active fragment, derivative, or analogue thereof;
(b) culturing said cells by suspending said cells in a medium which includes at least one agent that suppresses production of hyaluronic acid or hyaluronan or a salt thereof by said cells, wherein said cells are cultured for a time sufficient to express said biologically active protein or polypeptide, or a biologically active fragment, derivative, or analogue thereof; and
(c) recovering said expressed biologically active protein or polypeptide, or a biologically active fragment, derivative, or analogue thereof, from said cells.

13. The method of claim 12 wherein said mammalian cells are CHO cells.

14. The method of claim 12 wherein said biologically active protein or polypeptide is TSG-6 protein or a biologically active fragment, derivative, or analogue thereof.

15. The method of claim 14 wherein said TSG-6 protein or biologically active fragment, derivative, or analogue thereof has at least one histidine residue at the C-terminal thereof.

16. The method of claim 15 wherein said TSG-6 protein or biologically active fragment, derivative, or analogue thereof has 6 histidine residues at the C-terminal thereof.

17. The method of claim 12 wherein said at least one agent that suppresses production of hyaluronic acid or salt thereof by said cells is 4-methylumbelliferone.

18. The method of claim 1 wherein said medium further includes at least one agent that inhibits or prevents the aggregation of said cells.

19. The method of claim 18 wherein said at least one agent that inhibits or prevents the aggregation of said cells is selected from the group consisting of heparin, dextran sulfate, ferric citrate, and combinations thereof.

20. The method of claim 19 wherein said at least one agent that inhibits or prevents the aggregation of said cells is heparin.

21. A biologically active protein or polypeptide, or biologically active fragment, derivative, or analogue thereof produced by the method of claim 12.

22. A composition comprising:
(a) the biologically active protein or polypeptide, or biologically active fragment, derivative, or analogue thereof of claim 21; and
(b) an acceptable pharmaceutical carrier.
